(19) United States
(12) Patent Application Publication
(10) Pub. No.: US 2005/0176118 A1
(43) Pub. Date: Aug. 11, 2005

(54) Esterases with lipase activity

(76) Inventors: John Graham Oakeshott, Wanniassa (AU); Alan Devonshire, Harpenden (GB); Christopher Wayne Coppin, Ngunnawal (AU); Rama Heidari, Aharoo (AU); Susan Jane Dorrian, Fraser (AU); Robyn Joyce Russell, Wanniassa (AU)

Correspondence Address: GREENLEE WINNER AND SULLIVAN P C, 4875 PEARL EAST CIRCLE, SUITE 200, BOULDER, CO, 80301, US

(21) Appl. No.: 10/503,691

(22) PCT Filed: Feb. 6, 2002
(86) PCT No.: PCT/AU02/00113

(51) Int. Cl. C12P 7/64; C12P 13/00; C12P 7/62; C12N 9/20

(52) U.S. Cl. 435/128; 435/134; 435/135; 435/198

(57) ABSTRACT

The present invention relates to the use of insect esterases or lipases, or mutants thereof, as catalysts in biotransformation processes. The present invention may have application in any process involving hydrolysis, esterification, transesterification, interesterification or acylation reactions. The invention also has application in the enzymatic resolution of compounds to produce optically active compounds and has particular, but not exclusive, application to substrates having a hydrophobic moiety such as pyrethroids and fatty acid esters.

### FIELD OF THE INVENTION

The present invention relates to the use of lipases and esterases as catalysts in biotransformation processes. It is particularly concerned with the use of insect esterases and lipases, and mutants thereof, in such processes. The present invention may have application in any process involving hydrolysis, esterification, transesterification, interesterification or acylation reactions. The invention also has application in the enzymatic resolution of compounds to produce optically active compounds and has particular, but not exclusive, application to substrates having a hydrophobic moiety such as pyrethroids and fatty acid esters.

### BACKGROUND OF THE INVENTION

The industrial potential of lipases and esterases covers the range of their hydrolytic, esterification, transesterification and acylating activities. Comprehensive overviews of lipase- and esterase-catalysed industrial reactions can be found in Kazlauskas and Bornscheuer (1998), Phythian (1998), Anderson et al. (1998), Jaeger and Reetz (1998), Pandey et al. (1999) and Villeneuve et al. (2000), the disclosures of each of these references being incorporated herein in their entirety by reference.

Applications principally involving the hydrolytic activity of lipases and esterases cover substrates as diverse as triglycerides, aliphatic, alicyclic, bicyclic and aromatic esters and even esters based on organometallic sandwich compounds. Traditional uses include detergents for domestic and industrial applications. Other industrial applications include leather tanning, food processing (including fruit juices, baked foods, vegetable fermentation and dairy enrichment) and removal of pitch in the pulp produced in the paper industry. There are also now applications in the pharmaceutical and neutraceutical sectors, including various anti-obesity treatments. Biosensor applications are emerging as well, particularly for the determination of triacylglycerols in the medical field but also in the food and drink industry.

Of particular interest is the relatively recent use of the hydrolytic capability of lipases or esterases in various biotransformations to obtain novel and/or chiral building blocks or products for the fine chemical, pharmaceutical and agrochemical industries. Regio- and chiral purity is increasingly required of products in these industries. Total sales of therapeutics in 1995 was estimated to be US$150 billion, US$60 billion of which resulted from chiral compounds. Chiral drugs with sales volume exceeding US$1 billion include amoxycillin (an antibiotic), captopril (an angiotensin-converting-enzyme inhibitor) and erythropoietin (the haematopoietic growth factor). Often, just one of the enantiomers of a given pharmaceutical or agrochemical compound exerts the desired effect, but regulatory authorities are increasingly concerned to evaluate both/all chiral forms of all potential new drugs. Sometimes alternative forms may actually have undesirable side effects, as now appears to have been the case with thalidomide. Only about 25% of pharmaceuticals were enantiomerically pure in the 1990's but the industry projects that over half the new products in the next decades will need to be chirally pure.

An example of a use under consideration for the hydrolytic activity of these enzymes is a chiral biotransformation for the agrochemical industry involving pyrethroid insecticides, where the requisite quantities of the alcohol and acid building blocks of these carboxylester pesticides could be produced with high yields and high purity from racemic starting materials using enantiospecific hydrolyses (Hirohara and Nishizawa, 1998; Liese and Filho, 1999). Examples of such uses are described in U.S. Pat. Nos. 5,180,671, 4,985,364 and 6,207,429. Other examples where esterases or lipases can be used for kinetic resolution of ester racemates in the fine chemical or pharmaceutical industries involve phenylglycidyl ester (a precursor for diltiazem—a cardiovascular drug), glycidylbutyrate, and (1S-2S)-trans-2-methoxycyclohexanol for synthesis of β-lactam antibiotics of the Trinems type. A process for the enzymatic kinetic resolution of 3-phenylglycidates by enzyme catalysed transesterification with amino alcohols is described in U.S. Pat. No. 6,187,936, the disclosure of which is incorporated herein by cross-reference. U.S. Pat. No. 5,571,704, the disclosure of which is incorporated herein, describes the preparations of esters of (2R, 3S)-3-(4-methoxyphenyl)-glycidic acid by subjecting an enantiomeric mixture of the ester to enantiomeric enzyme transesterification in the presence of a lipase of animal or microbial origin in the presence of an alcohol which is different from the alcohol esterifying the acid. U.S. Pat. No. 5,750,382, the disclosure of which is also incorporated by reference, describes a process for producing optically active 2-alkoxycyclohexanols derivatives by treating a racemic mixture of the alcohol with a lipase in the presence of an acyl donor.

Significantly the chiral specificity of hydrolysis can be varied by varying usage of e.g. organic solvents and other reaction conditions. Thus a particular lipase may be used in reactions of very different chiral specificity (Rubio et al. (1991); Kazlauskas and Bomscheuer, (1998); Villeneuve et al. (2000), and Berglund (2001)).

Furthermore, with appropriate manipulation of organic solvent conditions the forward, hydrolysis, reaction is suppressed and the reverse esterification, reaction predominates (see, Villeneuve et al., 2000; Berglund 2001). Depending on the enzyme and conditions, this reverse reaction may or may not be regio- or chirally specific and there are important applications for both selective and non-selective esterifications.

As an example of non-regio-selective esterification the Candida albicans β-lipase (CALB) can be especially efficient in the preparation of homogeneous triglycerides. This is because it can acylate the secondary as well as the primary hydroxyls of glycerol to produce, for example, the long-chain omega-3 type polyunsaturated fatty acid triglycerides. Another application where homogenous products may be desirable involves production of biodiesel from esterification of various short chain alcohols with various fatty acids. See for example, U.S. Pat. Nos. 5,697,986 and 5,288,619, the disclosures of which are incorporated herein by cross-reference.

Recently, however, most attention has focussed on the uses of lipases and esterases for chemo-, regio- and stereo-selective esterification reactions. The importance of such selective synthesis for the pharmaceutical and neutraceutical fine chemical and agrochemical industries was noted in the discussion on esterase- and lipase-mediated hydrolysis reactions above. It is equally true for their esterification reactions. Enantioselective esterification is of interest both for use with chiral substrates and for the kinetic resolution of racemates. Significantly although individual enzymes will generally favour the same pro-chiral group in both the esterification and hydrolysis reactions, the two reactions can be used to generate opposite enantiomers. For example, acetylation of 2-benzyl glycerol by some lipases yields the (S)-monoacetate, while hydrolysis of the diacetate by the same enzymes yields the (R)-monoacetate, even though they react at the pro-R position in both cases (Kazlauskas and Bornscheuer (1998) and references therein).

One major limitation in the use of either the forward or reverse reaction for the kinetic resolution of racemates has been the fact that 50% conversion is the maximum possible. However methods are becoming available for improving efficiencies. Improvements based on mutagenesis to improve selectivity and novel immobilisation techniques to enhance activity and stability in organic solvents will be covered below. Another improvement involves dynamic kinetic resolution wherein a second catalyst is used to induce racemisation of the enantiomer not accepted by the enzyme. In some cases transition metal catalysts are used, which must be compatible with the lipase/esterase.

Transesterification refers to the process of exchanging acyl radicals between an ester and an acid (acidolysis), an ester and another ester (interesterification), or an ester and an alcohol (alcoholysis). There is considerable commercial interest in esterase and lipase-catalysed transesterification for the production of, for example, valuable food products. One case involves the production of dairy flavours in concentrated milks and creams. Another involves ester exchange to modify vegetable oils to high industrial qualities. Lever/Unilever has obtained a series of patents for the interesterification of fats and acylglycerols, for example U.S. Pat. Nos. 4,275,081 and 4,863,860, the disclosures of which are incorporated herein by reference. This process generates interesterified fats suitable for use in emulsions and other fat-based food products such as margarine, artificial creams and ice creams.

One interesting suite of applications of lipases/esterases that can exploit their hydrolytic, esterification or transesterification capabilities concerns the production of polymers. For example, polyesters can be produced by successive esterification and transesterification of di-functional esters and alcohols, self-condensation of di functional monomers, and ring opening polymerisation of lactones (Chaudhary et al. 1997 and references therein). U.S. Pat. No. 5,478,910, the disclosure of which is incorporated herein in its entirety by reference, describes a process for producing a polyester comprising reacting an organic diol with either an organic diester or an organic dicarboxylic acid in the presence of a supercritical fluid and in the presence of a solid esterase (preferably a lipase) enzyme. U.S. Pat. No. 5,962,624, the disclosure of which is also incorporated herein by reference, describes a process for making linear polyester by reacting polyols comprising at least two primary alcohol groups and at least one secondary alcohol or amino group and a dicarboxylic acid or a dicarboxylic acid ester in the presence of an effective amount of a lipase. The secondary OH or amino group of the polyol moiety is unreacted.

The potential of esterases and lipases as acylating agents derives from their two step reaction mechanism involving an acylated enzyme intermediate. In the case of the forward (hydrolysis) reaction, the reaction is just the acylation of water. For the backward (esterification) reaction it is the acylation of an alcohol. However many of these enzymes can acylate nucleophiles other than water, not necessarily containing oxygen, or esterify acyl donors other than alcohol. While focus historically has been on pro-chiral alcohols as acyl donors there is now interest in a much wider range of compounds including diols, α- and β-hydroxy acids and many others.

Candida albicans β-lipase illustrates many of the potentialities in respect of alternative acylation. Thus it will accept amino, hydroperoxy and thiol groups as nucleophiles instead of water or an alcohol and it can be used to prepare optically active amides or resolve chiral amines. Processes using this enzyme have been described for preparation of pure β-amino acids and R-amines. The enzyme will catalyse aminolysis with carboxylic esters, triglycerides, aryl esters, β-keto esters, α-β unsaturated esters and acryl esters. N-acyl amino acids and N-acyl amino acid amides have been made and there is also great potential for production of carbonates and carbamates. The latter in particular are of great value to the pharmaceutical industry. Whereas current chemical syntheses involves some notably toxic reagents, the lipase mediated synthesis uses, for example, vinyl or oxime carbonates.

Examples of acylation processes are: U.S. Pat. No. 5,210,030 which describes the selective acylation of immunomycin, by using an immobilised lipase, an acyl donor and a dry, non hydroxylic organic solvent; U.S. Pat. No. 5,387,514 which describes a method of acylation of alcohols using a vinyl ester and a lipase immobilised on a polystyrene resin; U.S. Pat. No. 6,261,813, which describes a method of derivatising a compound having hydroxyl groups by back to back acylation using a bifunctional acyl donor in the presence of a lipase to form an activated acyl ester or carbonate which is then used to acylate a nucleophile in the presence of a lipase; and U.S. Pat. No. 5,902,738 which describes the manufacture of a precursor for the production of Vitamin A by acylating a compound in the presence of an acylating agent, an organic solvent and a lipase.

Many of the useful reactions of lipases in particular depend on use of organic solvents where rates of catalysis can be slow. One solution to this has involved immobilisation on inorganic matrices like silica gel. This can be achieved by adsorption or covalent cross-linking. Alternatives to immobilisation include cross-linked enzyme crystals, reverse micelles and lipid- or surfactant-coated enzymes. The various alternatives are reviewed in (Kazlauskas and Bornscheuer, 1998; Villeneuve et al. 2000; and Berglund 2001).

Apart from manipulation of reaction conditions (‘solvent engineering’) there is also the possibility of altering enantioselectivity by genetic engineering. Two different approaches have been tested; site directed mutagenesis and in vitro evolution. The former relies on prior empirical or inferential knowledge of protein structure and substrate interactions to make mutations with predicted effects. This is often called rational design and in the case of esterases and lipases it is aided by empirical information of tertiary structures for over a dozen related carboxyl/cholinesterases and lipases. The latter does not necessarily use such prior information but allows for the accumulation by selection of multiple mutations enhancing the desired effects anywhere in the target gene/enzyme system, or region thereof. There are now a few examples of both approaches affecting the enantiospecificity of esterases/lipases (see Villeneuve et al. 2000; Svendsen 2000; and Berglund 2001 for reviews).

The best evidence for altered enantiospecificity by rational design involves a substrate binding site within the active site of the sn-1(3) regioselective Rhizopus oryzae lipase (ROL) (Scheib et al. 1998). Residue 258 in the hydrophobic patch of ROL that accommodates its sn2 substituent proved to be important for the stereospecificity of its hydrolysis of triradylglycerols, with a smaller effect attributed to residue 254, also in the same hydrophobic patch. In this case the empirical behaviour of the mutants closely matched the predicted behaviour from rational design principles. However in another example involving site directed mutagenesis the empirical behaviour differed from predictions. In this case (Hirose et al. 1995), involving Lipase PS from Pseudomonas cepacia, the stereospecificity of hydrolysis of 1,4 dihydropyridines was inverted in a triple mutant of sites 221, 266 and 287, although none of the individual mutations had marked effects.

Further evidence for altered enantiospecificity by in vitro evolution involves a Pseudomonas aeruginosa lipase (PAL) that is quite closely related to Lipase PS above (Liebeton et al. 2000). After four rounds of evolution a mutant was selected which had substantially altered enantioselectivity for the hydrolysis of the model substrate 2-methyldecanoic acid p-nitrophenol ester. The mutant enzyme had five different mutations, all well away from the substrate binding sites of the enzyme and the stereocentre of bound substrate. Instead they lay in, or close to, loops which are involved in the enzyme's transition from a ‘closed’ to an open ‘lid’ configuration at the lip of the active site.

A few esterases and rather more lipases are now in use industrially, however, as far as the present inventors are aware, none of these involve the use of insect esterases or lipases.

The dipteran α-carboxyl esterase cluster is a group of phylogenetically related genes in the carboxyl/cholinesterase multigene family that are also generally closely linked physically in the genome (Oakeshott et al., 1999). The cluster has been characterised molecularly in species of the higher Diptera from the genera Drosophila, Lucilia and Musca. It has attracted much interest over the last decade because mutations conferring OP insecticide resistance map to the cluster (Newcomb et al., 1997; Campbell et al., 1998; Claudianos et al., 1999). It forms a distinct sub-clade in phylogenetic analysis of the carboxyl/cholinesterase multigene family (FIG. 1). The only other members of its clade identified to date are other insect carboxylesterases mutants of which are also implicated in OP resistance (FIG. 1). These include genes/enzymes from lower Diptera (mosquitoes), Hemiptera (aphids) and Hymenoptera (wasps). It is likely therefore that this lade of carboxylesterases with at least about 30% identity to the Drosophila α-esterase cluster exists throughout the Insecta.

Little is known about the natural (i.e. non-OP insecticide) substrates of these carboxylesterases apart from their ability in vitro to hydrolyse simple, water-soluble, synthetic esters like methyl butyrate and naphthyl acetate that are widely taken as diagnostic of carboxylesterase activity. Their carboxyl esterase activity can be severely compromised in mutants that have acquired OP hydrolase activity.

The present inventors have now found that, surprisingly, insect esterases and lipases such as those in the α-carboxylesterase clade, and mutants thereof, also have activity against various large and hydrophobic carboxylesters, including fatty acid esters, for example, 4-methyl umbelliferyl palmitate as well as non-fatty acid hydrophobic molecules like pyrethroids.

### SUMMARY OF THE INVENTION

In a first aspect, the present invention provides an enzyme-based biocatalysis process, wherein the enzyme is an insect esterase or lipase, or a mutant thereof.

Lipases are generally considered to favour substrates with simple acid moieties and complex alcohol moieties whereas esterases are generally considered to favour substrates with complex acid and simple alcohol moieties (see, for example, Phythian, 1998). Insect esterases or lipases such as those in the α-carboxylesterase clade, and mutants thereof, are unusual in accommodating simple or complex acid or alcohol moieties. Thus, the insect esterases above, and mutants thereof, may be considered either esterases or lipases.

Furthermore, like some other lipases and esterases, these insect esterase and lipases show a high degree of regio- and stereo-specificity. Additionally, their regio- and stereo-specificity can be qualitatively altered by simple amino acid changes. These mutations can alter stereo-specificity in both their acid and alcohol groups. They are therefore potentially useful for a wide range of applications now being explored for lipase- or esterase-based biocatalysis.

In a preferred embodiment of the first aspect, the insect esterase or lipase is a member of the carboxyl/cholinesterase multi-gene family of enzymes. More preferably, the insect esterase or lipase is from the α-carboxylesterase clade within this multigene family (Oakeshott et al., 1999). Even more preferably, the insect esterase or lipase is a member of the α-carboxylesterase cluster which forms a sub-clade within this multi-gene family (Oakeshott et al., 1999) (FIG. 1). Esterases or lipase which form this sub-clade include at least α-carboxylesterases which can be isolated from species of Diptera, Hemiptera and Hymenoptera. Specific enzymes which are found in this sub-clade include, but are not limited to, the E3, EST23 or E4 esterases or lipases. However, orthologous of E3, EST23 or E4 from other insect species can also be used in the processes of the present invention.

Preferably, the α-carboxylesterase can be isolated from a species of Diptera. More preferably, the α-carboxylesterase cluster of higher Diptera from genera including Drosophila, Lucilia and Musca (Oakeshott et al., 1999). Accordingly, examples of preferred α-carboxylesterases for use in the present invention are the E3 esterase (SEQ ID NO:1) which is derived from Lucilia cuprina, or the EST23 esterase (SEQ ID NO:2) which is derived from Drosophila melanogaster.

In a further preferred embodiment, the mutant insect esterase or lipase has a mutation(s) in the oxyanion hole, acyl binding pocket or anionic site regions of the active site, or any combination thereof.

In a further preferred embodiment, the mutant α-carboxylesterase is selected from the group consisting of: E3G137R, E3G137H, E3W251L, E3W251S, E3W251G, E3W251T, E3W251A, E3W251L/F309L, E3W251L/G137D, E3W251L/P250S, E3F309L, E3Y148F, E3E217M, E3F354W, E3F354L, and EST23W251L. Preferably, the mutant α-carboxylesterase is E3W251L, E3F309L, E3W251L/F309L or EST23W251L.

In another preferred embodiment of the first aspect, the α-carboxylesterase, or mutant thereof, has a sequence selected from the group consisting of:
i) a sequence as shown in SEQ ID NO:1,
ii) a sequence as shown in SEQ ID NO:2,
iii) a sequence as shown in SEQ ID NO:3, and
iv) a sequence which is at least 40% identical to any one of i) to iii) which is capable of hydrolysing a hydrophobic ester. More preferably, the polypeptide is at least 50% identical, more preferably at least 60% identical, more preferably at least 70% identical, more preferably at least 80% identical, and more preferably at least 90% identical, more preferably at least 95% identical, and even more preferably at least 97% identical to any one of i) to iii).

The biocatalysis process of the invention may consist of or include the scheme: 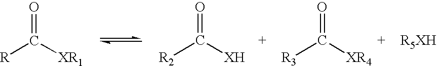
wherein
R, R₂ and R₃ are the same moiety Z, or
R is a mixture of stereoisomers of the moiety Z, R₂ is a stereoisomer of the moiety Z and R₃ is a mixture of stereoisomers enriched in another stereoisomer of moiety Z;
R₁, R₄ and R₅ are the same moiety Y, or
R₁ is a mixture of stereoisomers of the moiety Y, R₅ is one stereoisomer of the moiety and R₄ is a mixture of sterioisomers enriched in another stereoisomer of moiety Y;
Z and Y, which may be the same or different, may be any hydrocarbon moiety; and
X is a nucleophilic group.

Z and Y may be selected from the group consisting of:
substituted or unsubstituted, saturated or unsaturated straight-chain or branched acyclic or acyclic hydrocarbon optionally interrupted by one or more hetero atoms;
substituted or unsubstituted, saturated or unsaturated fused polycyclic hydrocarbons;
substituted or unsubstituted, saturated or unsaturated bridged hydrocarbons;
substituted or unsubstituted, saturated or unsaturated spiro hydrocarbons;
substituted or unsubstituted, saturated or unsaturated ring assemblies;
substituted or unsubstituted, saturated or unsaturated, bridged or unbridged heterocyclic ring system; and
substituted or unsubstituted, saturated or unsaturated, spiro or non-spiro, bridged or unbridged fused heterocyclic ring system.

Non-limiting examples of Z and Y are alphabeta unsaturated carbonyl, ketones, aldehydes, acids, aryloxys, phenols, cyano-s epoxides, alphahydroxyacids, amido, polyols, and amino acids.

Because there is an equilibrium, it is possible to select conditions in which either the forward reaction or the back reaction predominates.

The process of the invention may be carried out under conditions in which the forward reaction predominates.

In this case, the process of the invention may be used for chemo-, regio- or stereo-selective hydrolysis reactions. For example, the process may be used for resolution of a stereoisomer from a mixture of stereoisomers of a carboxylic acid ester. The stereoisomers may be enantiomers or positional stereoisomers.

In one particular embodiment, the process of the invention may be used for optical resolution of a mixture of a (R)-ester compound and a (S)-ester compound comprising the steps of:
(a) contacting an insect esterase or lipase, or mutant thereof, with the mixture to obtain an optically active compound or an optically active alcohol compound by stereoselectively hydrolyzing one of the (R)-ester compound and the (S)-ester compound; and
(b) recovering an optically active compound selected from the group consisting of the optically active acid compound, the optically active alcohol compound and the optically active ester that is not hydrolysed.

The process may be carried out so that the backward reaction predominates in which case the process of the invention may be used for the acylation of a compound R₅XH, where R₅ and X are as defined above.

In this case, the process of the invention may be used for chemo-, regio- or stereo-selective esterification reactions. For example, it may be used to produce an optically active ester using pure or racemic mixtures of the starting compounds, ie ester and R₅XH. The stereoisomers may be enantiomers or positional stereoisomers.

The process of the invention may also be a transesterification reaction, for example, as represented generally as follows:

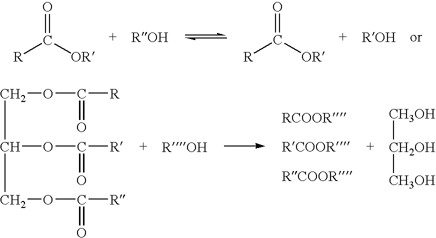

The process of the invention may be an interesterification reaction (ester interchange) for example, as represented generally as follows:

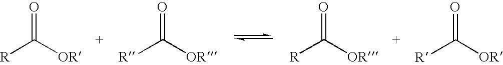

The process may be carried out on a substrate that is an ester having a hydrophilic and/or hydrophobic moieties. The ester may be a hydrophobic carboxylester. The hydrophobic moiety may be in the acid and/or alcohol residue of the ester. The hydrophobic portion may be, for example, a C₃ to C₃₆ or more hydrocarbons. The hydrophobic moiety may be a moiety containing hydrophobic ring groups such as one or more carbocylic rings, which may be saturated or unsaturated. The hydrophobic moiety may be the residue of a pyrethroid alcohol.

The process of the invention may be used to produce an optically active acid or alcohol from a mixture of optical isomers. In the case of the optical resolution of an acid, the substrate may be a simple ester of the acid, e.g. C₁-C₄ akyl ester of the acid. In the case of the optical resolution of an alcohol, the substrate may be a simple ester of the alcohol, e.g. C₁-C₄ akyl ester of the alcohol. The acid may be a substituted or unsubstituted cyclopropanecarboxylic acid. The alcohol may be a substituted or unsubstituted phenoxybenzyl alcohol. For example, the process of the invention may be used to produce an optical isomer of a pyrethroid acid or a pyrethroid alcohol used to synthesise pyrethroid pesticides. Pyrethroids are synthetic analogues of the natural pyrethrins, which are produced in the flowers of the pyrethrum plant (Tanacetum cinerariifolium). Modification of their structure has yielded compounds that retain the intrinsically modest vertebrate toxicity of the natural products but are both more stable and more potent as pesticides. The pyrethroid may be a Type I pyrethroid or a Type II pyrethroid, Pyrethroids Type I pyrethroid compounds (e.g., permethrin) differ from Type II pyrethroid compounds in that Type II compounds possess a cyano group on the α-carbon atom of the phenoxybenzyl moiety.

Examples of pyrethroids include, but are not restricted to these compounds; permethrin, cyloprothrin, fenvalerate, esfenvalerate, flucythrinate, fluvalinate, fenpropathrin, d-fenothrin, cyfenothrin, allethrin, cypermethrin, deltamethrin, tralomethrin, tetramethrin, resmethrin and cyfluthrin.

The process of the present invention has wide application including those applications discussed above under the heading “Background to the Invention” above, wherein an insect esterase or lipase, or mutant thereof, is used as the catalyst.

Thus the process of the present invention has application in those applications involving the use of esterases or lipases including:
detergents for domestic and industrial applications; leather tanning; food processing (including fruit juices, baked foods, vegetable fermentation and dairy enrichment);
removal of pitch in the pulp produced in the paper industry;
pharmaceutical/neutraceutical sectors and in biosensor applications are emerging as well, particularly for the determination of triacylglycerols in the medical field and food and drink industry;
biotransformations to obtain novel and/or chiral building blocks or products for the fine chemical, pharmaceutical and agrochemical industries, particularly those based on regio- and chiral purity;
chiral biotransformation for the agrochemical industry involving pyrethroid insecticides, where the requisite quantities of the alcohol and acid building blocks of these carboxylester pesticides;
esterase and lipase-catalysed transesterification for the production of eg valuable food products including dairy flavours in concentrated milks and creams;
ester exchange to modify vegetable oils to high industrial qualities, including interesterified fats suitable for use in emulsions and other fat-based food products such as margarine, artificial creams and ice creams;
production of polymers, for example, polyesters can be produced by successive esterification and transesterification of di functional esters and alcohols, self-condensation of di functional monomers, and ring opening polymerisation of lactones;
production of biofuels including biodeisel; and
acylation reactions.

Preferably, the process is performed in a liquid containing environment.

The insect esterase or lipase, or mutant thereof, may be provided by any appropriate means. This includes providing the insect esterase or lipase, or mutant thereof, directly with or without carriers or excipients etc. The insect esterase or lipase, or mutant thereof, can also be provided in the form of a host cell such a transformed prokaryote or eukaryote cell, typically a microorganism such as a bacterium or a fungus, which expresses a polynucleotide encoding the insect esterase or lipase, or mutant thereof.

The insect esterase or lipase, or mutant thereof, can also be as provided a polymeric sponge or foam, the foam or sponge comprising the insect esterase or lipase, or mutant thereof, immobilized on a polymeric porous support.

Preferably, the porous support comprises polyurethane.

In a preferred embodiment, the sponge or foam further comprises carbon embedded or integrated on or in the porous support.

It is envisaged that the use of a surfactant in the process of the present invention may liberate potential substrates, particularly those which are hydrophobic from any, for example, sediment in the sample. Thus increasing efficiency of the process of the present invention. Accordingly, in another preferred embodiment, the process comprises the presence of a surfactant More preferably, the surfactant is a biosurfactant.

In another aspect, the present invention provides a method for generating and selecting an enzyme that hydrolyses a hydrophobic ester, the method comprising
(i) introducing one or more mutations into an insect esterase or lipase, or an insect esterase or lipase that has already been mutated, and
(ii) determining the ability of the mutant insect esterase or lipase to hydrolyse the hydrophobic ester.

Preferably, the hydrophobic ester is a fatty acid ester.

Preferably, the one or more mutations enhances hydrolytic activity and/or alters the stereospecificity of the esterase or lipase.

Preferably, the insect esterase or lipase is an α-carboxylesterase.

Preferably, the α-carboxylesterase has a sequence selected from the group consisting of:
i) a sequence as shown in SEQ ID NO:1,
ii) a sequence as shown in SEQ ID NO:2,
iii) a sequence as shown in SEQ ID NO:3, and
iv) a sequence which is at least 40% identical to any one of i) to iii). More preferably, the sequence is at least 50% identical, more preferably at least 60% identical, more preferably at least 70% identical, more preferably at least 80% identical, and more preferably at least 90% identical, more preferably at least 95% identical, and even more preferably at least 97% identical to any one of i) to iii).

Preferably, the one or more mutations are within a region of the esterase or lipase is selected from the group consisting of: oxyanion hole, acyl binding pocket and anionic site.

Preferably, the mutation is a point mutation.

Preferably, the insect esterase or lipase that has already been mutated is selected from the group consisting of: E3G137R, E3G137H, E3W251L, E3W251S, E3W251G, E3W251T, E3W251A, E3W251L/F309L, E3W251L/G137D, E3W251L/P250S, E3F309L, E3Y148F, E3E217M, E3F354W, E3F354L, and EST23W251L.

In another aspect, the present invention provides a method for generating and selecting an insect α-carboxylesterase that hydrolyses an ester, the method comprising
(i) introducing one or more mutations into an insect α-carboxylesterase, or an insect α-carboxylesterase that has already been mutated, and
(ii) determining the ability of the mutant insect α-carboxylesterase to hydrolyse the ester.

Preferably, the one or more mutations enhances hydrolytic activity and/or alters the stereospecificity of the insect α-carboxylesterase.

In a further aspect, the present invention provides an enzyme obtained by a method according to the two previous aspect.

The invention is hereinafter described by way of the following non-limiting example and with reference to the accompanying figures.

### BRIEF DESCRIPTION OF THE ACCOMPANYING DRAWINGS

FIG. 1: Phylogeny of the carboxyl/cholinesterase multigene family (Oakeshott et al. 1999). Most of the sequences for the 140 proteins analysed can be found in the Pfam, C. elegans (http://www.sanger.ac.uk/Projects/C_elegans/blast_server.shtml) and COG NCBI databases. Key references are given in Oakeshott et al. (1999). Sequences were aligned using the Pileup program of the Genetics Computer Group (GCG), with default settings (gap weight 3.0 and gap length weight 0.1). Terminal lineages containing multiple paralogous sequences are indicated by (•). A full presentation of the phylogeny for 49 sequences in the C. elegans database is also given in Oakeshott et al. (1999). CE=carboxylesterase. The vertebrate CES1-CES4 groups are those of Satoh and Hosokawa (1998).

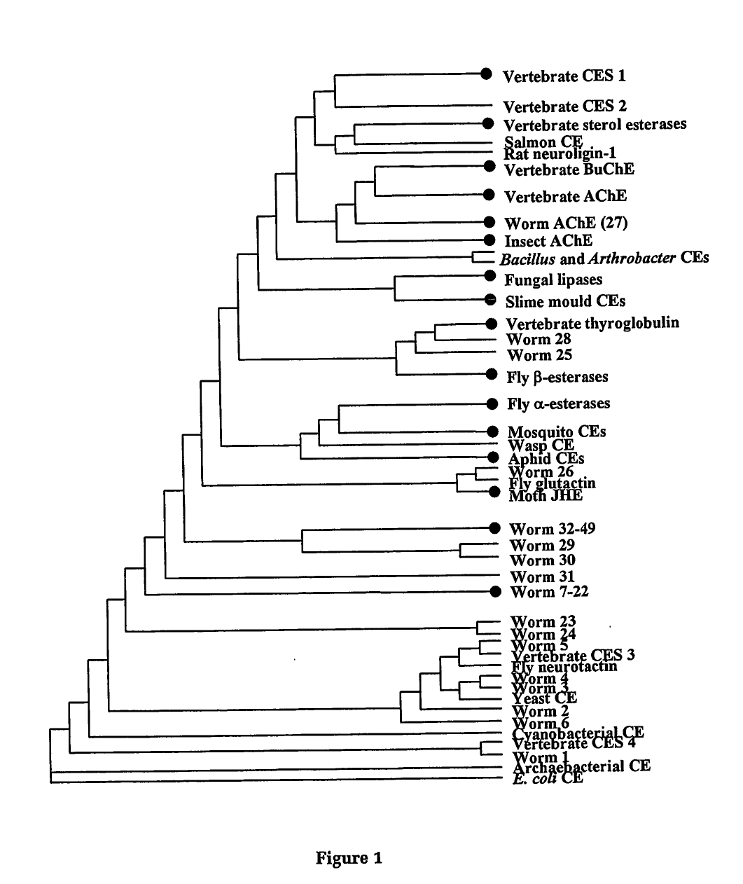

FIG. 2: Amino acid sequence alignment of the E3 (SEQ ID NO:1) and Torpedo californica acetylcholinesterase (SEQ ID NO:4) enzymes. The sequence around the active site serine and residues Gly137, Trp251 and Phe309 are shown in bold and underlined.

FIG. 3: Proposed configuration of active site of LcE3 carboxylesterase in an acylation reaction.

FIG. 4: Results of representative titration experiments performed on cell extracts containing baculovirus expressed esterases.

FIG. 5: Molecular structures for 1R/S cis and trans permethrin, 1R/S cis and trans NRDC157 and the four stereoisomers of cis deltamethrin.

FIG. 6: Hydrolysis of cis and trans permethrin (0.5 μM by E3W251L.

### KEY TO SEQUENCE LISTING

SEQ ID NO:1—Amino acid sequence of Lucilia cuprina E3 α-carboxylesterase.
SEQ ID NO:2—Amino acid sequence of Drosophila melanogaster EST23 α-carboxylesterase.
SEQ ID NO:3—Amino acid sequence of Myzus persicae E4 α-carboxylesterase.
SEQ ID NO:4—Partial amino acid sequence of Torpedo califonica acetylcholinesterase.

### DETAILED DESCRIPTION OF THE INVENTION

General Techniques

Unless otherwise indicated, the recombinant DNA techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (Editors), Current Protocols in Molecular Biology, Greene Pub. Associates and Wiley-Interscience (1988, including all updates until present) and are incorporated herein by reference.

Definitions

In this specification the term “substituted” includes substitution by a group which may or may not be further substituted with one or more groups selected from alkyl, cycloalkyl, alkenyl, alkynyl, aryl, arylalkyl, halo, haloalkyl, haloalkynyl, hydroxy, alkoxy, alkenyloxy, haloalkoxy, haloalkenyloxy, nitro, amino, nitroalkyl, nitroalkenyl, nitroalkynyl, nitroheterocyclyl, alkylamino, dialkylamino, alkenylamine, alkynylamino, acyl, alkenacyl, alkynylacyl, acylamino, diacylamino, acyloxy, alkylsulfonyloxy, heterocyclyl, heterocycloxy, heterocyclamino, haloheterocyclyl, alkylsulfenyl, carboalkoxy, alkylthio, acylthio, phosphorus-containing groups such as phosphono and phosphinyl.

The term “alkyl” as used herein is taken to mean both straight chain alkyl groups such as methyl, ethyl, propyl, isopropyl, butyl, isobutyl, sec-butyl, tertiary butyl, and the like. The alkyl group may optionally be substituted by one or more groups selected from alkyl, cycloalkyl, alkenyl, alkynyl, halo, haloalkyl, haloalkynyl, hydroxy, alkoxy, alkenyloxy, haloalkoxy, haloalkenyloxy, nitro, amino, nitroalkyl, nitroalkenyl, nitroalkynyl, nitroheterocyclyl, alkylamino, dialkylamino, alkenylamine, alkynylamino, acyl, alkenoyl, alknoyl, acylamino, diacylamino, acyloxy, alkylsulfonyloxy, heterocyclyl, heterocycloxy, heterocyclamino, haloheterocyclyl, alkylsulfenyl, alkylcarbonyloxy, alkylthio, acylthio, phosphorus-containing groups such as phosphono and phosphinyl.

The term “alkoxy” as used herein denotes straight chain or branched alkyloxy, preferably C₁₋₁₀ alkoxy. Examples include methoxy, ethoxy, n-propoxy, isopropoxy and the different butoxy isomers.

The term “alkenyl” as used herein denotes groups formed from straight chain, branched or mono- or polycyclic alkenes and polyene. Substituents include mono- or poly-unsaturated alkyl or cycloalkyl groups as previously defined, preferably C₂₋₁₀ alkenyl. Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1-4,pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl, or 1,3,5,7-cyclooctatetraenyl.

The term “halogen” as used herein denotes fluorine, chlorine, bromine or iodine, preferably bromine or fluorine.

The term “heteroatoms” as used herein denotes O, N or S.

The term “acyl” used either alone or in compound words such as “acyloxy”, “acylthio”, “acylamino” or diacylamino” denotes an aliphatic acyl group and an acyl group containing a heterocyclic ring which is referred to as heterocyclic acyl, preferably a C₁₋₁₀ alkanoyl. Examples of acyl include carbamoyl; straight chain or branched alkanoyl, such as formyl, acetyl, propanoyl, butanoyl, 2-methylpropanoyl, pentanoyl, 2,2-dimethylpropanoyl, hexanoyl, heptanoyl, octanoyl, nonanoyl, decanoyl; alkoxycarbonyl, such as methoxycarbonyl, ethoxycarbonyl, t-butoxycarbonyl, t-pentyloxycarbonyl or heptyloxycarbonyl; cycloalkanecarbonyl such as cyclopropanecarbonyl cyclobutanecarbonyl, cyclopentanecarbonyl or cyclohexanecarbonyl; alkanesulfonyl, such as methanesulfonyl or ethanesulfonyl; alkoxysulfonyl, such as methoxysulfonyl or ethoxysulfonyl; heterocycloalkanecarbonyl; heterocyclyoalkanoyl, such as pyrrolidinylacetyl, pyrrolidinylpropanoyl, pyrrolidinylbutanoyl, pyrrolidinylpentanoyl, pyrrolidinylhexanoyl or thiazolidinylacetyl; heterocyclylalkenoyl, such as heterocyclylpropenoyl, heterocyclylbutenoyl, heterocyclylpentenoyl or heterocyclylhexenoyl; or heterocyclylglyoxyloyl, such as, thiazolidinylglyoxyloyl or pyrrolidinylglyoxyloyl.

Insect Esterases, Lipases, and Mutants Thereof

The % identity of a polypeptide is determined by GAP (Needleman and Wunsch, 1970) analysis (GCG program) with a gap creation penalty=5, and a gap extension penalty=0.3. The query sequence is at least 15 amino acids in length, and the GAP analysis aligns the two sequences over a region of at least 15 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. More preferably, the query sequence is at least 250 amino acids in length and the GAP analysis aligns the two sequences over a region of at least 250 amino acids. Even more preferably, the query sequence is at least 500 amino acids in length and the GAP analysis aligns the two sequences over a region of at least 500 amino acids.

As used herein, the term “mutant thereof” refers to mutants of a naturally occurring insect esterase or lipase which maintains at least some hydrolytic activity towards an ester-containing compound as described herein when compared to the naturally occurring insect esterase or lipase from which they are derived. Preferably, the mutant has enhanced activity and/or altered stereospecificity when compared to the naturally occurring insect esterases or lipases from which they are derived.

Amino acid sequence mutants of naturally occurring insect esterases or lipases can be prepared by introducing appropriate nucleotide changes into a nucleic acid of the present invention, or by in vitro synthesis of the desired polypeptide. Such mutants include, for example, deletions, insertions or substitutions of residues within the amino acid sequence. A combination of deletion, insertion and substitution can be made to arrive at the final construct, provided that the final protein product possesses the desired characteristics.

In designing amino acid sequence mutants, the location of the mutation site and the nature of the mutation will depend on characteristic(s) to be modified. In a particularly preferred embodiment, naturally occurring insect esterases or lipases are mutated to increase their ability to hydrolyse an ester-containing compound as described herein. The sites for mutation can be modified individually or in series, e.g., by (1) substituting first with conservative amino acid choices and then with more radical selections depending upon the results achieved, (2) deleting the target residue, or (3) inserting other residues adjacent to the located site. Examples of such mutants include; E3G137R, E3G137H, E3W251L, E3W251S, E3W251G, E3W251T, E3W251A, E3W251L/F309L, E3W251L/G137D, E3W251L/P250S, E3F309L, E3Y148F, E3E217M, E3F354W, E3F354L, and EST23W251L.

Mutants useful for the processes of the present invention can also be obtained by the use of the DNA shuffling technique (Patten et al., 1997). DNA shuffling is a process for recursive recombination and mutation, performed by random fragmentation of a pool of related genes, followed by reassembly of the fragments by primerless PCR. Generally, DNA shuffling provides a means for generating libraries of polynucleotides which can be selected or screened for, in this case, polynucleotides encoding enzymes which can hydrolyse an ester-containing compound as described herein. The stereospecificity of the selected enzymes can also be screened.

Amino acid sequence deletions generally range from about 1 to 30 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as the active or binding site(s). Other sites of interest are those in which particular residues obtained from various strains or species are identical. These positions may be important for biological activity. These sites, especially those falling within a sequence of at least three other identically conserved sites, can be substituted in a relatively conservative manner. Such conservative substitutions are shown in Table 1 under the heading of “exemplary substitutions”.

Furthermore, if desired, unnatural amino acids or chemical amino acid analogues can be introduced as a substitution or addition into the insect esterase or lipase, or mutants thereof. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

TABLE 1

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

Also included within the scope of the invention are insect esterases or lipases, or mutants thereof, which are differentially modified during or after synthesis, e.g., by biotinylation, benzylation, glycosylation, acetylation, phosphorylation, derivatization by known protecting/blocking groups, proteolytic cleavage, linkage to an antibody molecule or other cellular ligand, etc. These modifications may serve to increase the stability and/or bioactivity of the polypeptide of the invention.

Insect esterases or lipases, or mutants thereof, can be produced in a variety of ways, including production and recovery of natural proteins, production and recovery of recombinant proteins, and chemical synthesis of the proteins. In one embodiment, an isolated polypeptide encoding the insect esterase or lipase, or mutant thereof, is produced by culturing a cell capable of expressing the polypeptide under conditions effective to produce the polypeptide, and recovering the polypeptide. A preferred cell to culture is a recombinant cell of the present invention. Effective culture conditions include, but are not limited to, effective media, bioreactor, temperature, pH and oxygen conditions that permit protein production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells producing the insect esterase or lipase, or mutant thereof, can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Recombinant Vectors

Recombinant vectors can be used to express an insect esterase or lipase, or mutant thereof, for use in the proceses of the present invention. In addition, in another embodiment of the present invention includes a recombinant vector, which includes at least one isolated polynucleotide which encodes an insect esterase or lipase, or mutant thereof, inserted into any vector capable of delivering the polynucleotide molecule into a host cell. Such vectors contain heterologous polynucleotide sequences, that is polynucleotide sequences that are not naturally found adjacent to polynucleotide encoding the insect esterase or lipase, or mutant thereof, and that preferably are derived from a species other than the species from which the esterase or lipase is derived. The vector can be either RNA or DNA, either prokaryotic or eukaryotic, and typically is a virus or a plasmid.

One type of recombinant vector comprises a polynucleotide encoding an insect esterase or lipase, or mutant thereof, operatively linked to an expression vector. The phrase operatively linked refers to insertion of a polynucleotide molecule into an expression vector in a manner such that the molecule is able to be expressed when transformed into a host cell. As used herein, an expression vector is a DNA or RNA vector that is capable of transforming a host cell and of effecting expression of a specified polynucleotide molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, endoparasite, arthropod, other animal, and plant cells. Preferred expression vectors of the present invention can direct gene expression in bacterial, yeast, arthropod and mammalian cells and more preferably in the cell types disclosed herein.

Expression vectors of the present invention contain regulatory sequences such as transcription control sequences, translation control sequences, origins of replication, and other regulatory sequences that are compatible with the recombinant cell and that control the expression of polynucleotide molecules of the present invention. In particular, expression vectors which comprise a polynucleotide encoding an insect esterase or lipase, or mutant thereof, include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, arthropod and mammalian cells, such as, but not limited to, tac, lac, trp, trc, oxy-pro, omp/lpp, rrnB, bacteriophage lambda, bacteriophage T7, T7lac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells. Additional suitable transcription control sequences include tissue-specific promoters and enhancers.

Polynucleotide encoding an insect esterase or lipase, or mutant thereof, may also (a) contain secretory signals (i.e., signal segment nucleic acid sequences) to enable an expressed insect esterase or lipase, or mutant thereof, to be secreted from the cell that produces the polypeptide and/or (b) contain fusion sequences. Examples of suitable signal segments include any signal segment capable of directing the secretion of an insect esterase or lipase, or mutant thereof. Preferred signal segments include, but are not limited to, tissue plasminogen activator (t-PA), interferon, interleukin, growth hormone, histocompatibility and viral envelope glycoprotein signal segments, as well as natural signal sequences. In addition, polynucleotides encoding an insect esterase or lipase, or mutant thereof, can be joined to a fusion segment that directs the encoded protein to the proteosome, such as a ubiquitin fusion segment.

Host Cells

Another embodiment of the present invention includes a recombinant cell comprising a host cell transformed with one or more polynucleotides encoding an insect esterase or lipase, or mutant thereof. Transformation of a polynucleotide molecule into a cell can be accomplished by any method by which a polynucleotide molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. A transformed polynucleotide encoding an insect esterase or lipase, or mutant thereof, can remain extrachromosomal or can integrate into one or more sites within a chromosome of the transformed (i.e., recombinant) cell in such a manner that their ability to be expressed is retained.

Suitable host cells to transform include any cell that can be transformed with a polynucleotide encoding an insect esterase or lipase, or mutant thereof. Host cells of the present invention either can be endogenously (i.e., naturally) capable of producing an insect esterase or lipase, or mutant thereof, or can be capable of producing such proteins after being transformed with at least one polynucleotide encoding an insect esterase or lipase, or mutant thereof. Host cells of the present invention can be any cell capable of producing at least one insect esterase or lipase, or mutant thereof, and include bacterial, fungal (including yeast), parasite, arthropod, animal and plant cells. Preferred host cells include bacterial, mycobacterial, yeast, arthropod and mammalian cells. More preferred host cells include Salmonella, Escherichia, Bacillus, Listeria, Saccharomyces, Spodoptera, Mycobacteria, Trichoplusia, BHK (baby hamster kidney) cells, MDCK cells (normal dog kidney cell line for canine herpesvirus cultivation), CRFK cells (normal cat kidney cell line for feline herpesvirus cultivation), CV-1 cells (African monkey kidney cell line used, for example, to culture raccoon poxvirus), COS (e.g., COS-7) cells, and Vero cells. Particularly preferred host cells are E. coli, including E. coli K-12 derivatives; Salmonella typhi; Salmonella typhimurium, including attenuated strains; Spodoptera frugiperda; Trichoplusia ni; BHK cells; MDCK cells; CRFK cells; CV-1 cells; COS cells; Vero cells; and non-tumorigenic mouse myoblast G8 cells (e.g., ATCC CRL 1246). Additional appropriate mammalian cell hosts include other kidney cell lines, other fibroblast cell lines (e.g., human, murine or chicken embryo fibroblast cell lines), myeloma cell lines, Chinese hamster ovary cells, mouse NIH/3T3 cells, LMTK cells and/or HeLa cells.

Recombinant DNA technologies can be used to improve expression of a transformed polynucleotide molecule by manipulating, for example, the number of copies of the polynucleotide molecule within a host cell, the efficiency with which those polynucleotide molecules are transcribed, the efficiency with which the resultant transcripts are translated, and the efficiency of post-translational modifications. Recombinant techniques useful for increasing the expression of a polynucleotide encoding an insect esterase or lipase, or mutant thereof, include, but are not limited to, operatively linking polynucleotide molecules to high-copy number plasmids, integration of the polynucleotide molecule into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotide molecules of the present invention to correspond to the codon usage of the host cell, and the deletion of sequences that destabilize transcripts.

Compositions

Compositions useful for the processes of the present invention, or which comprise an insect esterase or lipase, or mutant thereof, include excipients, also referred to herein as “acceptable carriers”. An excipient can be any material that is suitable for use in the processes described herein. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal or o-cresol, formalin and benzyl alcohol. Excipients can also be used to increase the half-life of a composition, for example, but are not limited to, polymeric controlled release vehicles, biodegradable implants, liposomes, bacteria, viruses, other cells, oils, esters, and glycols.

Furthermore, the insect esterase or lipase, or mutant thereof, can be provided in a composition which enhances the rate and/or degree of biocatalysis, or increases the stability of the polypeptide. For example, the insect esterase or lipase, or mutant thereof, can be immobilized on a polyurethane matrix (Gordon et al., 1999), or encapsulated in appropriate liposomes (Petrikovics et al. 2000a and b). The insect esterase or lipase, or mutant thereof, can also be incorporated into a composition comprising a foam such as those used routinely in fire-fighting (Lejeune et al., 1998).

As would be appreciated by the skilled addressee, the insect esterase or lipase, or mutant thereof, could readily be used in a sponge or foam as disclosed in WO 00/64539, the contents of which are incorporated herein in their entirety.

The concentration of the insect esterase or lipase, or mutant thereof, (or host cell expressing the insect esterase or lipase, or mutant thereof) that will be required to produce effective biocatalysis will depend on a number of factors including the nature of the reaction that needs to be performed, and the formulation of the composition. The effective concentration of the insect esterase or lipase, or mutant thereof, (or host cell expressing the insect esterase or lipase, or mutant thereof) within a composition can readily be determined experimentally, as will be understood by the skilled artisan.

Surfactants

It is envisaged that the use of a surfactant in the processes of the present invention may liberate potential substrates, particularly those which are hydrophobic from any, for example, sediment in a sample. Thus increasing efficiency of the processes of the present invention.

Surfactants are amphipathic molecules with both hydrophilic and hydrophobic (generally hydrocarbon) moieties that partition preferentially at the interface between fluid phases and different degrees of polarity and hydrogen bonding such as oil/water or air/water interfaces. These properties render surfactants capable of reducing surface and interfacial tension and forming microemulsion where hydrocarbons can solubilize in water or where water can solubilize in hydrocarbons. Surfactants have a number of useful properties, including dispersing traits.

Biosurfactants are a structurally diverse group of surface-active molecules synthesized by microorganisms. These molecules reduce surface and interfacial tensions in both aqueous solutions and hydrocarbon mixtures. Biosurfactants have several advantages over chemical surfactants, such as lower toxicity, higher biodegradability, better environmental comparability, higher foaming, high selectivity and specificity at extreme temperatures, pH and salinity, and the ability to be synthesized from a renewable source.

Biosurfactants useful in the biotransformation processes of the present invention include, but are not limited to; glycolipids such as rhamnolipids (from, for example, Pseudomonas aeruginosa), trehalolipids (from, for example, Rhodococcus erythropolis), sophorolipids (from, for example, Torulopsis bombicola), and cellobiolipids (from, for example, Ustilago zeae); lipopeptides and lipoproteins such as serrawettin (from, for example, Serratia marcescens), surfactin (from, for example, Bacillus subtilis); subtilisin (from, for example, Bacillus subtilis), gramicidins (from, for example, Bacillus brevis), and polymyxins (from, for example, Bacillus polymyxa); fatty acids, neutral lipids, and phospholipids; polymeric surfactants such as emulsan (from, for example, Acinetobacter calcoaceticus), biodispersan (from, for example, Acinetobacter calcoaceticus), mannan-lipid-protein (from, for example, Candida tropicalis), liposan (from, for example, Candida lypolytica), protein PA (from, for example, Pseudomonas. aeruginosa); and particulate biosurfactants such as vesicles and fimbriae from, for example, A. calcoaceticus.

### EXAMPLES

### Example 1

### Construction of Mutants

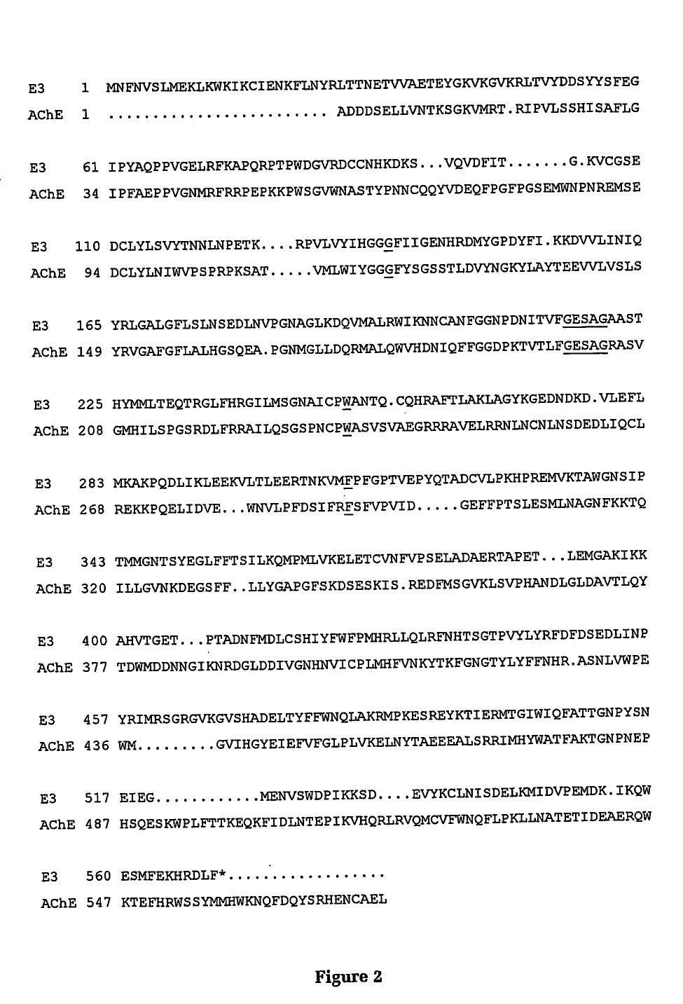

An alignment of the amino acid sequence of the E3 enzyme with that of a vertebrate acetylcholinesterase (TcAChE, for which the three dimensional structure is known; Sussman et al., 1991) is given in FIG. 2. Mutants of E3 and EST23 were constructed using the QuickChange™ Site-Directed Mutagenesis Kit of Stratagene and are named according to the number of the residue that has been changed, and the nature of that change. For example, mutant E3W251L is an E3 mutant in which the Trp residue at position 251 in the wild-type enzyme (i.e. E3WT) has been mutated to Leu.

E3 and EST23 enzymes were expressed using the baculovirus expression system as described by Newcomb et al. (1997), but using the HyQ SFX-insect serum-free medium (HyClone) for increased expression. Cell extracts were prepared by lysing the cells at a concentration of 10⁸ cells ml⁻¹ in 0.1M phosphate buffer pH 7.0 containing 0.05% Triton X-100. Extracts were then titrated for the number of esterase molecules using a fluorometric assay based on the initial release of coumarin (a fluorescent compound) upon phosphorylation of the enzyme by diethylcoumaryl phosphate (dECP).

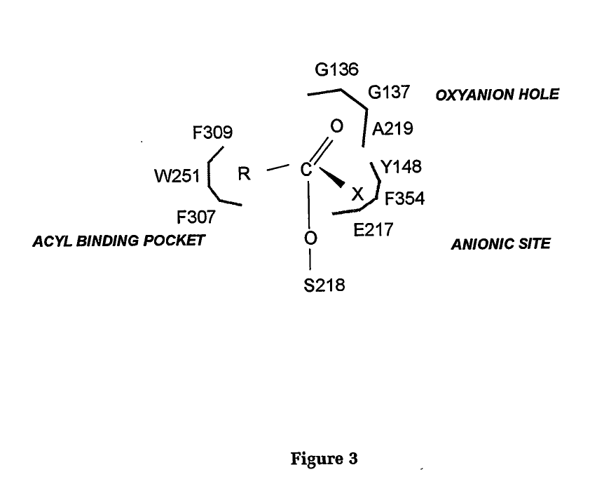

FIG. 3 illustrates the proposed configuration of the active site of E3 (based on the three dimensional structure of vertebrate AChE) in an acylation reaction. We have examined mutations in seven E3 residues in regions corresponding to three distinct subsites of the known AChE active site.

These are the oxyanion hole (E3 residue 137), the anionic site (E3 residues 148, 217 and 354) and acyl binding pocket (E3 residues 250, 251 and 309). The anionic site and acyl binding pocket correspond to the p1 and p2 subsites in the nomenclature of Jarv (1984).

Mutations in the Oxyanion Hole

In TcAChE the oxyanion hole comprises Gly118, Gly119 and Ala201, which corresponds to Gly136, Gly137 and Ala219 in E3. These residues are highly conserved throughout the carboxyl/cholinesterase multigene family (Oakeshott et al., 1999) and there is empirical evidence for the conservation of the oxyanion hole structure from X-ray crystallographic studies of several cholinesterases and lipases (Cygler and Schrag, 1997), albeit the structure does change during interfacial activation in some lipases (Derewenda et al., 1992). There is also empirical structural evidence for their function in stabilising the oxyanion formed by the carbonyl oxygen of the carboxylester substrate as the first transition state during catalysis (Grochulski et al., 1993; Martinez et al., 1994). This stabilisation is achieved by a network of hydrogen bonds to the amide groups of the three key residues in the peptide chain (Ordentlich et al., 1998). Recently Koellner et al. (2000) have also shown that both Gly residues in the AChE oxyanion hole make hydrogen bonds with buried “structural” water molecules, which are retained during catalysis and thought to act as lubricants to facilitate traffic of substrates and products within the active site.

Three further mutations were made to the Gly137 of E3 in addition to the G137D found naturally in OP resistant L. cuprina. First, Glu was substituted as the other acidic amino acid, in G137E. The mutant G137H was also constructed, because His is also non-protonated at neutral pH (pKₐ about 6.5 cf 4.4 for Asp and Glu) and it was found to confer some OP hydrolysis on human butyrylcholinesterase when substituted for either Gly in its oxyanion hole (Broomfield et al., 1999). Finally, Arg (pKₐ around 12) was substituted at position 137, to examine the effects of the most strongly basic substitution possible.

Mutations in the Acyl Binding Pocket

The acyl binding pockets of structurally characterised cholinesterases are formed principally from four non-polar residues, three of which are generally also aromatic. Together they create a strongly hydrophobic pocket to accommodate the acyl moiety of bound substrate. The four residues in TcAChE are Trp233, Phe288, Phe290 and Val400 corresponding to Trp251, Val307, Phe309 and Phe422 in E3. Similar arrays of hydrophobic residues appear to be conserved at the corresponding sites of most carboxyl/cholinesterases (Oakeshott et al., 1993; Robin et al., 1996; Yao et al., 1997; Harel et al., 2000). In particular Trp is strongly conserved at residue 233/251 and 290/309 is Phe in cholinesterases and most carboxylesterases, albeit a Leu or Ile in several lipases and a few carboxylesterases. The residue corresponding to TcAChE Phe288 is typically a branched chain aliphatic amino acid in cholinesterases that show a preference for longer chain esters such as butyrylcholine. This includes mammalian butyrylcholinesterase and some insect acetylcholinesterases, which have a butyrylcholinesterase-like substrate specificity. The branched chain aliphatic amino acid appears to provide a greater space in the acyl-binding pocket to accommodate the larger acyl group.

Mutational studies of 288/307 and 290/309 in several cholinesterases confirm their key role in determining aspects of substrate specificities related to acyl group identity. In human AChE replacement of the Phe at either position with a smaller residue like Ala improves the kinetics of the enzyme for substrates like propyl- or butyl-(thio)choline with larger acyl groups than the natural acetyl(thio)choline substrate (Ordentlich et al., 1993). In AChE from D. melanogaster and the housefly, Musca domestica, natural mutations of their 290/309 equivalent to the bulkier, polar Tyr that contributes to target site OP resistance have lower reactivity to both acetylcholine and OPs (Fournier et al., 1992; Walsh et al., 2001). For D. melanogaster AChE, substitution of this Phe residue with the smaller Leu gave the predicted increase in OP sensitivity, although surprisingly replacement with other small residues like Gly, Ser or Val did not (Vilatte et al., 2000).

Trp 233/251 has received much less attention in mutational studies of cholinesterases but our prior work on E3 shows its replacement with a smaller Leu residue again increases reactivity for carboxylester substrates with bulky acyl moieties, or for OPs (Campbell et al., 1998a, b; Devonshire et al., 2002). A mutation to Gly has also been found in a homologue from the wasp, Anisopteromalus calandrae, that shows enhanced malathion carboxylesterase (MCE) kinetics (Zhu et al., 1999) while a Ser has been found in a homologue from M. domestica that may be associated with malathion resistance (Claudianos et al., 2002). In respect of OP hydrolase activity Devonshire et al. (2002) proposed that the particular benefit of such mutations is to accommodate the inversion about the phosphorus that must occur for the second hydrolysis stage of the reaction to proceed. Notably Devonshire et al. (2002) found that the k_{cat} for OP hydrolase activity of E3W251L is an order of magnitude higher for dMUP, with its smaller dimethyl phosphate group than for dECP, which has a diethyl phosphate group. This suggests that there remain tight steric constraints on the inversion even in a mutant with a larger acyl pocket.

We have mutated both the W251 and F309 residues of E3 as well as the P250 immediately adjacent to W251. In addition to the previously characterised natural W251L mutation we have now analysed substitutions with four other small amino acids in W251S, W251G, W251T and W251A. A double mutant of W251L and P250S was also analysed, because a natural variant of the ortholog of E3 in M. domestica with high MCE activity has Ser and Leu at positions 250 and 251, respectively. Only one F309 substitution was examined, F309L, which the AChE results suggest should enhance MCE and OP hydrolyse activities. F309L was analysed alone and as a double mutant with W251L.

Mutations in the Anionic Site

The anionic site of cholinesterases is sometimes called the quaternary binding site (for the quaternary ammonium in acetylcholine), or the p1 subsite in the original nomenclature of Jarv (1984). It principally involves Trp 84, Glu 199 and Phe 330, with Phe 331 and Tyr 130 (TcAChE nomenclature) also involved. Except for Glu 199 it is thus a highly hydrophobic site. Glu 199 is immediately adjacent to the catalytic Ser 200. The key residues are highly conserved across cholinesterases and to a lesser extent, many carboxylesterases (Oakeshott et al., 1993; Ordentlich et al., 1995; Robin et al., 1996; Claudianos et al., 2002). Except for Trp 84 (the sequence alignment in FIG. 2 shows that E3 is missing residues corresponding to AChE residues 74-85), E3 has identical residues to TcAChE at the corresponding positions (217, 354 and 148, respectively). Interestingly the equivalent of Glu 199 is Gln and the equivalent of the Phe 330 is Leu in some lipases and certain carboxylesterases, whose substrates are known to have small leaving groups (Thomas et al., 1999; Campbell et al., 2001; Claudianos et al., 2002).

Structural and mutational studies have provided a detailed picture of the role of the anionic site in cholinesterase catalysis. The key residues form part of a hydrogen bonded network at the bottom of the active site, with Tyr 130 and Glu 199 also sharing contact with a structural water molecule (Ordentlich et al., 1995; Koellner et al., 2000). The anionic site undergoes a conformational change when substrate binds a peripheral binding site at the lip of the active site gorge, the new conformation accommodating the choline (leaving) group of the substrate and facilitating the interaction of its carbonyl carbon with the catalytic Ser 200 (Shafferman et al., 1992; Ordentlich et al., 1995; 1996). Consequently the site functions mainly in the first, enzyme acylation, stage of the reaction and, in particular, in the formation of the non-covalent transition state (Nair et al., 1994). Therefore mutations of the key residues mainly affect Kₘ rather than k_{cat}. The interactions with the choline leaving group are mainly mediated through non-polar and π-electron interactions, principally involving Trp 84 and Phe 330 (Ordentlich et al., 1995).

Studies with OP inhibitors suggest that the anionic site of cholinesterases also accommodates their leaving group but there is some evidence that part of the site (mainly Glu 199 and Tyr 130; also possibly Ser 226) may also then affect the reactivity of the phosphorylated enzyme (Qian and Kovach, 1993; and see also Ordentlich et al., 1996; Thomas et al., 1999).

There has been little mutational analysis of carboxylesterase sites corresponding to the AChE anionic site among but one interesting exception involves the EST6 carboxylesterase of D. melanogaster, which has a His at the equivalent of Glu 199. A mutant in which this His is replaced by Glu shows reduced activity against various carboxylester substrates but has acquired some acetylthiocholine hydrolytic activity (Myers et al., 1993). The E4 carboxylesterase of the aphid, Myzus persicae, has a Met at this position and this enzyme is unusually reactive to OPs (Devonshire and Moores, 1982). However, it is not known whether the Met contributes to the OP hydrolase activity. Similarly, a Y148F substitution is one of several recorded in the E3 ortholog in an OP resistant strain (ie also G137D) of M. domestica but it is not known whether this change directly contributes to OP hydrolase activity (Claudianos et al., 1999).

The Y148, E217 and F354 residues in E3 have now been mutated. E217M and Y148F mutations were made to test whether the corresponding mutations in the M. persicae and M. domestica enzymes above contribute directly to their OP reactivity. Y148F is also tested in a G137D double mutant since this is the combination found in the resistant M. domestica. F354 was mutated both to a smaller Leu residue and a larger Trp, Leu commonly being found at this position in lipases (see above).

### Example 2

### Enzyme Titrations

Four 100 μl reactions were set up for each expressed esterase in microplate columns 1-4:
plate well blank containing 0.025% Triton X-100, 0.1M phosphate buffer pH 7.0;
substrate blank containing 100 μM dECP in 0.025% Triton X-100, 0.1M phosphate buffer pH 7.0;
cell blank containing 50 μl cell extract mixed 1:1 with 0.1M phosphate buffer pH 7.0;
titration reaction containing 50 μl cell extract mixed 1:1 with 0.1M phosphate buffer pH 7.0 containing 200 μM dECP.

All components except dECP (freshly prepared at a concentration of 200 μM in buffer) were placed in the wells. Several enzymes were assayed simultaneously in a plate, and the reactions were started by adding dECP simultaneously to the 2nd and 4th wells down a column. The interval to the first reading (typically 1 minute) was noted for the subsequent calculations.

The mean value for the plate well blank (A) was subtracted from all readings before further calculations. Preliminary experiments with various cell extracts showed that they gave some fluorescence at 460 nm and that their addition to solutions of the assay product, 7-hydroxycoumarin, quenched fluorescence by 39(±7)%. Fluorescence values in the titration reactions (D) were therefore corrected for this quenching effect after subtraction of the intrinsic fluorescence of the cell extracts (C). Finally, the substrate blank (B), taken as the mean from all the simultaneous assays in a plate, was subtracted to give the corrected fluorescence caused by the esterase-released coumarin. These corrections were most important for cell lines expressing esterase at very low level (<1 pmol/μl extract).

The fully corrected data were plotted as a progress curve, and the equilibrium slope extrapolated back to zero time to determine the amount of esterase, based on its stoichiometric interaction with the inhibitor (the 100 μM concentration of dECP gave full saturation of the esterase catalytic sites of all these enzymes in 10-20 minutes). A calibration curve for 7-hydroxycoumarin was prepared alongside the reactions in all plates, and used to calculate molar concentration of enzyme and product formation.

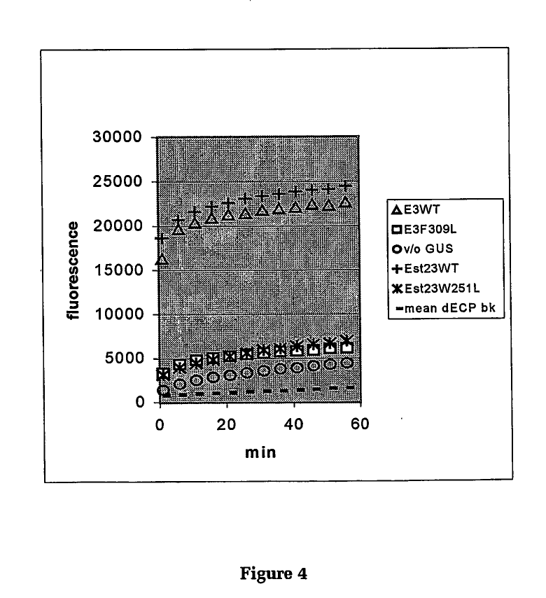

FIG. 4 shows the results of representative titration experiments performed on cell extracts containing baculovirus expressed esterases.

### Example 3

### Permetirin Hydrolysis Assays

Expressed enzymes were tested for permethrin hydrolytic activity using a radiometric partition assay for acid-labelled compounds, or a TLC based assay for those labelled in the alcohol moiety (Devonshire and Moores, 1982). Features of the assays include keeping the concentration of permethrin below its published solubility in aqueous solution (0.5 μM), the concentration of detergent (used to extract the enzyme from the insect cells in which it is expressed) below the critical micelle concentration (0.02% for Triton X100), and performing the assays quickly (ie within 10-30 minutes) to minimise the substrate sticking to the walls of the assay tubes (glass tubes were used to minimise stickiness). At these permethrin concentrations the enzyme is not saturated by the substrate, so Kₘ values could not be determined. However, specificity constants (k_{cat}/Kₘ) could be calculated accurately for each of the enzymes with permethrin activity, which allows direct comparison of their efficiency at low substrate concentrations. The power of the analyses was increased by separating permethrin into its cis and trans isomers.

(a) Separation of cis and trans Isomers of Permethrin

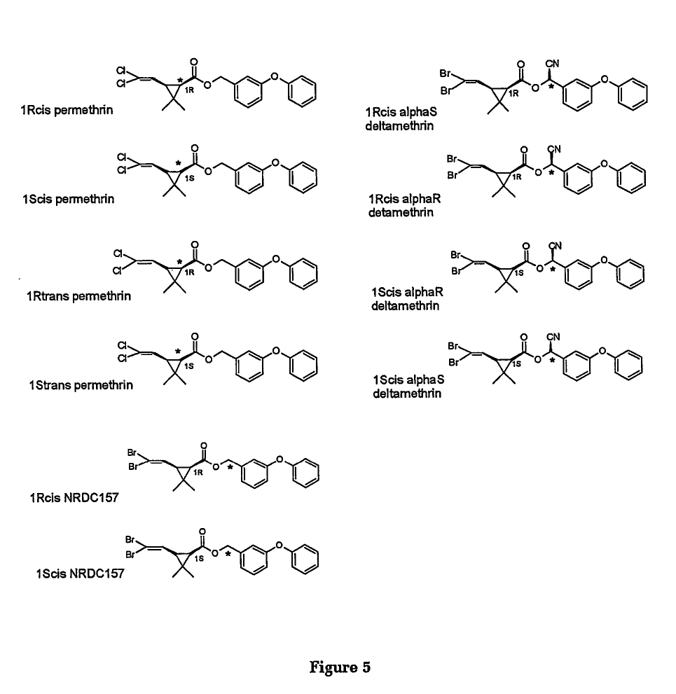

Commercial preparations of permethrin contain four stereoisomers: 1S cis, 1R cis, 1S trans, 1R trans (FIG. 5). Preparative thin layer chromatography (TLC) on silica was used to separate the isomers into two enantiomer pairs: 1S/1R cis and 1S/1R trans. The enantiomers could not be separated further. Enzyme preparations could then be assayed for the hydrolysis of each enantiomer pair.

(b) Assay Protocol

Pyrethroids Radiolabelled in the Acid Moiety

This assay (Devonshire and Moores, 1982) is used for permethrin isomers. It relies on incubating the expressed esterase with radiolabelled substrate and then measuring the radioactive cyclopropanecarboxylate anion in the aqueous phase after extracting the unchanged substrate into organic solvent. Based on previous experience, the best extraction protocol utilises a 2:1 (by volume) mixture of methanol and chloroform. When mixed in the appropriate proportion with aliquots of the assay incubation, the consequent mixture of buffer, methanol and chloroform is monophasic, which serves the purpose of stopping the enzyme reaction and ensuring the complete solubilization of the pyrethroid. Subsequent addition of an excess of chloroform and buffer exceeds the capacity of the methanol to hold the phases together, so that the organic phase can be removed and the product measured in the aqueous phase. In detail, the protocol is as follows.

Phosphate buffer (0.1M, pH 7.0) was added to radiolabelled permethrin (50 μM in acetone) to give a 1 μM solution and the assay then started by adding an equal volume of expressed esterase appropriately diluted in the same buffer. Preliminary work had established that the concentration of detergent (Triton X-100 used to extract esterase from the harvested cells) in the incubation had to be below its CMC (critical micelle concentration of 0.02%) to avoid the very lipophilic pyrethroid partitioning into the micelles and becoming unavailable to the enzyme. Typically, the final volume of the assay was 500-1000 μl, with substrate and acetone concentrations 0.5 μM and 1%, respectively. At intervals ranging from 30 seconds to 10 minutes at a temperature of 30°, 100 μl aliquots of the incubation were removed, added to tubes containing 300 μl of the 2:1 methanol chloroform mixture and vortex-mixed. The tubes were then held at room temperature until a batch could be further processed together, either at the end of the incubation or during an extended sampling interval. After adding 50 μl buffer and 100 μl chloroform, the mixture was vortex-mixed, centrifuged and the lower organic phase removed with a 500 μl Hamilton syringe and discarded. The extraction was repeated after adding a further 100 μl chloroform, and then 200 μl of the upper aqueous phase was removed (using a pipettor with a fine tip) for scintillation counting. It is critical to avoid taking any of the organic phase. Since the final volume of the aqueous phase was 260 μl (including some methanol), the total counts produced in the initial 100 μl aliquot were corrected accordingly.

Pyrethroids Radiolabelled in the Alcohol Moiety

i) Type I Pyrethroids—Dibromo Analogues (NRDC157) of Permethrin:

The 3-phenoxbenzyl alcohol formed on hydrolysis of these esters does not partition into the aqueous phase in the chloroform methanol extraction procedure. It was therefore necessary to separate this product from the substrate by TLC on silica (Devonshire and Mooers, 1982). In detail, the protocol is as follows.

Incubations were set up as for the acid-labelled substrates. The reactions were stopped at intervals in 100 μl aliquots taken from the incubation by immediately mixing with 200 μl acetone at −79° (solid CO₂). Then 100 μl of the mixture was transferred, together with 3 μl non-radioactive 3-phenoxbenzyl alcohol (2% in acetone), on to the loading zone of LinearQ channelled silica F254 plates (Whatman). After developing in a 10:3 mixture of toluene (saturated with formic acid) with diethyl ether, the substrate and product were located by radioautography for 6-7 days (confirming an identical mobility of the product to the cold standard 3-phenoxbenzyl alcohol revealed under UV light). These areas of the TLC plate were then impregnated with Neatan (Merck) and dried, after which they were peeled from the glass support and transferred to vials for scintillation counting. The counts were corrected for the 3-fold dilution of the initial 100 μl by acetone before spotting on the silica.

ii) Type II Pyrethroids—Deltamethrin Isomers:

Preliminary experiments, in which incubations were analysed by TLC as above, showed primarily the formation of 3-phenoxbenzoic acid, in line with literature reports that the initial cyanohydrin hydrolyis product is rapidly converted non-enzymically to the acid. Since the TLC assay is more protracted than the chloroform-methanol extraction procedure, the latter (as described above for acid-labelled pyrethroids) was adopted to measure the 3-phenoxbenzoate anion produced from these substrates.

For all assays the molar amount of product formed was calculated from the known specific activity of the radiolabelled substrate. Early experiments on the expressed E3WT esterase showed that the rate of hydrolysis was directly proportional to the concentration of 1RS cis or 1RS trans permethrin in the assay up to 0.5 μM, i.e. there was no accumulation of Michaelis complex. Assays at concentrations greater than 0.5 μM, which approximates the published aqueous solubility of permethrin, gave erratic results so precluding the measurement of Kₘ and k_{cat}. Furthermore, with the racemic substrates, the rate of hydrolysis slowed dramatically once approximately 50% of the substrate had been hydrolysed, indicating that only one of the two enantiomers (1R or 1S present in equal amounts in a racemic mixture) was readily hydrolysed, in line with previously published data for an esterase from aphids (Devonshire and Moores, 1982). Assay conditions were therefore adjusted to measure the hydrolysis of the more-readily hydrolysed enantiomer in each pair. Sequential incubation of trans permethrin with E3WT and E4 from OP resistant aphids (Myzus pericae) homogenates confirmed that both showed preference for the 1S trans enantiomer. In all cases, the rate of hydrolysis at 0.5 μM (or 0.25 μM for the one enantiomer in racemic substrates), together with the molar amount of esterase determined by titration with dECP, were used to calculate the specificity constant (k_{cat}/Kₘ) since it was not possible to separate these kinetic parameters. The same considerations about substrate solubility and proportionality of response to its concentration were assumed for all enzymes and substrates.

(c) Calculation of Specificity Constants

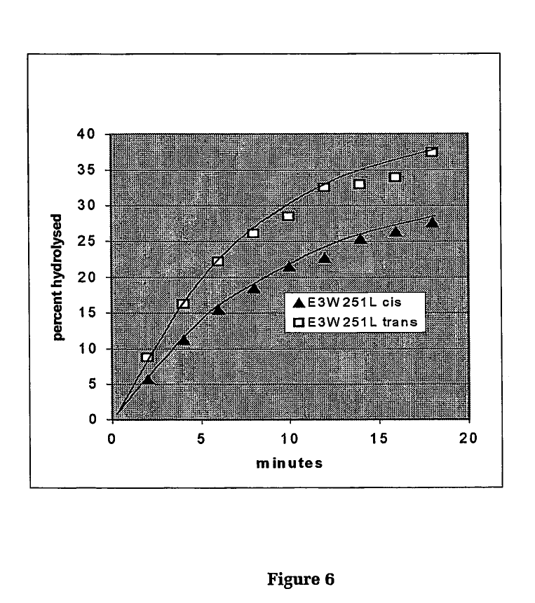

FIG. 6 presents the results of an experiment in which the trans- and cis-isomers of permethrin were hydrolysed by the E3W251L enzyme.

Since the rate of hydrolysis of permethrin isomers was directly proportional to the concentration of substrate used up to 0.5 μM (i.e. there was no significant formation of Michaelis complex), it was not possible to measure Kₘ and k_{cat} as independent parameters. At concentrations well below the Kₘ, the Michaelis-Menten equation simplifies to:

$$v = \frac{{k}_{cat}}{{K}_{m}} [ S ] [ E ]$$

The specificity constant (ie k_{cat}/Kₘ) can therefore be calculated from the above equation using the initial hydrolysis rate (pmol/min, calculated from the known specific activity of the radiolabelled substrate) and the concentrations of substrate and enzyme in the assay. The diffusion-limited maximum value for a specificity constant is 10⁸-10⁹ M⁻¹sec⁻¹ (Stryer, 1981).

### Example 4

### Permethrin Hydrolytic Activity of E3, EST23 and Myzus E4 Variants

Table 2 summarises the kinetic data obtained for eighteen E3, three EST23 and five MpE4 variants using cis- and trans-permethrin as substrates. In each case the data represent the hydrolysis of the enantiomer that is hydrolysed the fastest out of each of the 1S/1R cis and 1S/1R trans isomer pairs (see above).

The E3WT enzyme found in OP susceptible blowflies, its EST23 D. melanogaster orthologue and MpE4WT enzyme showed significant levels of permethrin hydrolytic activity, which was specific for the trans isomers. Mutations in either the acyl binding pocket or anionic site regions of the active site of the E3 enzyme resulted in significant increases in activity for both the trans and cis isomers of permethrin.

a) Oxyanion Hole Mutations

The E3G137D mutation is responsible for diazinon resistance in the sheep blowfly. In this mutant the very small, aliphatic, neutral Gly residue in the oxyanion hole region of the active site of the enzyme is replaced by an acidic Asp, allowing hydrolysis of a bound oxon OP molecule. However, this mutant (as well as its D. melanogaster orthologue and the corresponding MpE4G113D mutant) had reduced activity for trans-permethrin in particular, compared to that of the wild-type enzyme. This activity was not increased by substitution of Gly-137 with either His or Glu. However, substitution of Gly-137 with Arg did not affect the activity for either cis- or trans-permethrin appreciably. The linear nature of Arg might mean that it can fold easily and not interfere with binding of permethrin to the active site.

b) Acyl Binding Pocket Mutations

The E3W251L mutation, which replaces the large aromatic Trp reside with the smaller aliphatic Leu in the acyl pocket of the active site, resulted in a 7-fold increase in trans-permethrin hydrolysis and the acquisition of substantial cis-permethrin hydrolysis. The effect of W251L in EST23 was essentially the same as for E3. However, the corresponding W224L mutation in MpE4 resulted in a substantial decrease in activity for both trans- and cis-permethrin, due presumably to differences in the protein backbone. Replacement of Trp-251 with even smaller residues in E3 (Thr, Ser, Ala and Gly in decreasing order of size) also resulted in an increase in permethrin hydrolytic activity, although the activity of these mutants was not as high as that of E3W251L. Clearly, steric factors are not the only consideration in the activity of the mutants. For example, Thr and Ser both contain hydroxyl groups and are hydrophilic. Furthermore, Ala is both aliphatic and hydrophobic (like Leu) and even smaller than Leu, yet this mutant was as active for permethrin as the W251L mutant. Opening up the oxyanion hole of the W251L mutant (ie E3P250S/W251L) also decreased its activity for both cis- and trans-permethrin, although the activity was still higher than that of the wild type. It is interesting to note that increases in specificity constants for permethrin for all W251 mutants in E3 as well as W251L in EST23 compared to those of the wild types were uniformly more pronounced for the cis isomers. Whereas the wild type enzymes yielded trans:cis ratios of at least 20:1, these ratios were only 2-6:1 for the W251 mutants. The extra space in the acyl pocket provided by these mutants was apparently of greatest benefit for the hydrolysis of the otherwise more problematic cis isomers.

Combination of both the W251L and G137D mutations on to the same E3 molecule increased the activity of the enzyme for cis permethrin over wild-type levels, but decreased the activity for trans-permethrin. However, the activity of the double mutant was not as great as that of the mutant containing the E3W251L mutation alone (i.e. the mutations did not act additively).

Some lipases are known to have a Leu residue at the position corresponding to Phe 309 in L. cuprina E3. The E3F309L mutant was therefore constructed with the aim of conferring activity for lipophilic substrates like pyrethroids. As can be seen from Table 2, the E3F309L mutant was much better than E3WT for both isomers. It was even more active for trans-permethrin than E3W251L, though not as active for the cis isomers. Combination of both the F309L and W251L mutations on the same E3 molecule increased the activity for cis-permethrin and decreased the activity for trans-permethrin to E3W251L levels. In other words, the F309L mutation had very little effect on the activity of the W251L mutant for permethrin.

c) Anionic Site Mutations

Some lipases are known to have a Leu residue at the position corresponding to Phe 354 in L. cuprina E3. However, substitution of Phe 354 for Leu in E3 did not increase its activity for permethrin appreciably. Substitution of Phe 354 for the bulkier aromatic residue, Trp, on the other hand, increased activity for both cis- and trans-permethrin 3-4-fold. It is perhaps surprising that F354W, not F354L, should show increases in activity against the very lipophilic permethrin, given that it is a Leu that replaces Phe in some naturally occurring lipases.

The Y148F mutation produced large effects on permethrin kinetics and the effects were opposite in direction depending on genetic background. As a single mutant compared to wild type it shows 5-6 fold enhancement of activity for both cis and trans permethrin. As a double mutant with G137D (which as a single mutant gives values much lower than wild type), it shows a further two fold reduction for trans permethrin and and almost obliterates activity for cis permethrin. These latter results clearly imply a strong interaction of Y148 with the oxyanion hole in respect of permethrin hydrolysis.

Glu-217, the residue immediately adjacent to the catalytic serine, is thought to be important in stabilising the transition state intermediate in hydrolysis reactions. However, mutating this residue to Met (E3E217M), as found naturally in the esterase E4 of the aphid M. persicae, had little effect on permethrin activity. The converse mutation in MpE4 (ie MpE4M190E), however, decreased the activity of the MpE4 enzyme for both trans- and cis-permethrin by about half. Combining this mutation with the oxyanion hole mutation (MpE4G113D/M190E) resulted in a further substantial decrease in permethrin hydrolytic activity (ie the two mutations were additive in their effects on permethrin activity).

TABLE 2
Specificity constants of natural and synthetic variants of
L. cuprina esterase E3, D. melanogaster EST23 and Myzus E4
for the cis- and trans-isomers of permethrin, and the two
cis-dibromovinyl analogues of permethrin (NRDC157). Ratios of
the specificity constants for trans and cis permethrin, and
for 1S cis and 1R cis NRDC157 are also indicated.

| | Specificity Constant (k_{cat}/Kₘ M⁻¹sec⁻¹) | | | | | |
|---|---|---|---|---|---|---|
| | | 1S/1R cis- | | | | |
| | 1S/1R | permethrin | | | NRDC157 | |
| | trans- | (trans:cis | | NRDC157 | 1R cis | |
| Enzyme | permethrin | ratio) | | 1S cis | (1S:1R ratio) | |
| E3WT | 90 000 | 3 400 | (27:1) | 4 700 | 630 | (8:1) |

Oxyanion hole mutants:

| | | | | | |
|---|---|---|---|---|---|
| E3G137D | 9 600 | 1 800 | (5:1) | ND¹ | ND |
| E3G137R | 85 000 | 3 900 | (22:1) | ND | ND |
| E3G137H | 26 000 | 1 600 | (16:1) | ND | ND |
| E3G137E | 2 400 | 280 | (9:1) | ND | ND |

Acyl binding pocket mutants:

| | | | | | | |
|---|---|---|---|---|---|---|
| E3W251L | 900 000 | 460 000 | (2:1) | 370 000 | 5 400 | (68:1) |
| E3W251S | 140 000 | 36 000 | (4:1) | 35 000 | 2 900 | (12:1) |
| E3W251G | 95 000 | 24 000 | (4:1) | 27 000 | 1 700 | (16:1) |
| E3W251T | 150 000 | 24 000 | (6:1) | 24 000 | 900 | (26:1) |
| E3W251A | 300 000 | 72 000 | (4:1) | 67 000 | 1 200 | (56:1) |
| E3F309L | 1 200 000 | 48 000 | (25:1) | 5 700 | 8 000 | (0.7:1) |
| E3W251L/F309L | 810 000 | 430 000 | (2:1) | 26 000 | 69 100 | (0.4:1) |
| E3W251L/G137D | 24 000 | 11 000 | (2:1) | 12 000 | 1 100 | (11:1) |

| | | | | | |
|---|---|---|---|---|---|
| E3P250S/W251L | 340 000 | 110 000 | (3:1) | ND | ND |

Anionic site mutants:

| | | | | | |
|---|---|---|---|---|---|
| E3Y148F | 580 000 | 17 000 | (34:1) | ND | ND |
| E3Y148F/G137D | 4 100 | 47 | (87:1) | ND | ND |
| E3E217M | 93 000 | 4 400 | (21:1) | ND | ND |
| E3F354W | 350 000 | 8 800 | (40:1) | ND | ND |
| E3F354L | 104 400 | 2 700 | (38:1) | ND | ND |

EST23 enzymes:

| | | | | | | |
|---|---|---|---|---|---|---|
| EST23WT | 21 000 | 890 | (24:1) | 990 | 330 | (3:1) |
| EST23W251L | 260 000 | 160 000 | (2:1) | 72 000 | 1 200 | (60:1) |

| | | | |
|---|---|---|---|
| EST23G137D | 2 500 | ND | ND |

M. persicae E4 enzymes:

| | | | | | |
|---|---|---|---|---|---|
| MpE4WT | 270 000 | 2 400 | (113:1) | ND | ND |
| MpE4G113D | 12 000 | 830 | (14:1) | ND | ND |
| MpE4W224L | 23 000 | 1 100 | (21:1) | ND | ND |
| MpE4M190E | 120 000 | 1 200 | (100:1) | ND | ND |
| MpE4G113D/M190E | 6 300 | 210 | (30:1) | ND | ND |

¹Not determined
²Not substantially different from values obtained using control cell extracts

### Example 5

### Hydrolysis of Bromo-Permethrin Analogue

Table 2 also summarises the kinetic data obtained for the E3 and EST23 variants using the two cis-dibromovinyl analogues of permethrin (NRDC157). The 1S cis isomer of this dibromo analogue of permethrin was hydrolysed with similar efficiency to the 1R/1S cis permethrin by all enzymes except E3F309L and F309L/W251L. This indicates that the larger bromine atoms did not substantially obstruct access of this substrate to the catalytic centre. Although the activities with the E3WT and EST23WT enzymes were too low for significant comparison between isomers, all other enzymes except E3F309L and F309L/W251L showed 10 to 100-fold faster hydrolysis of the 1S isomer. This is the same preference for this configuration at C1 of the cyclopropane ring as found previously for 1S trans permethrin in M. persicae (Devonshire and Moores, 1982).

F309L showed a dramatic effect on NRDC157 kinetics. The single mutant showed little difference from wild type for 1S cis and the double with W251L showed less activity than W251L alone for this isomer. However, the 1S/1R preference was reversed, with values of 0.7:1 in the single mutant and 0.4:1 in the double. The result is the two highest values for 1R cis activities in all the data set. The value for the double mutant is in fact about 10 fold higher than those for either mutant alone.

### Example 6

### Hydrolysis of Type II Pyrethroids by Expressed Enzymes

Table 3 summarises the kinetic data obtained for a sub-set of the E3 and EST23 variants using the four deltamethrin cis isomers. With the exception of E3W251L and E3F309L, the 1R cis isomers of deltamethrin (whether αS or αR) were hydrolysed with similar efficiency to the 1R cis NRDC157 (which can be considered intermediate in character between permethrin and deltamethrin in that it has dibromovinyl substituent but lacks the α cyano group). Activity against 1R cis isomers was always greater with the αR than the αS conformation. E3W251L and E3F309L were markedly less efficient with the 1R cis isomers of deltamethrin than with the corresponding isomers of NRDC157.

TABLE 3
Specificity constants for the four deltamethrin cis isomers

| | Specificity Constant (k_{cat}/Kₘ M⁻¹sec⁻¹) | | | |
|---|---|---|---|---|
| | 1S cis αR | 1S cis αS | 1R cis αR | 1R cis αS |
| Enzyme | deltamethrin | deltamethrin | deltamethrin | deltamethrin |
| E3WT | —¹ | — | — | — |
| E3G137D | — | — | 890 | 560 |
| E3G137R | — | — | 670 | 350 |
| E3G137H | ND | ND | ND | ND |
| E3G137E | ND | ND | ND | ND |
| E3W251L | 990 | 880 | 380 | — |
| E3W251S | 4 600 | 2 460 | ND² | ND |
| E3W251G | 700 | 170 | 690 | 350 |
| E3W251T | 2 900 | 520 | 2 100 | 1 300 |
| E3W251A | 2 000 | 660 | 1 300 | 730 |
| E3F309L | 2 400 | 810 | 1 600 | 840 |
| E3W251L/ | 3 600 | 410 | 2 700 | 1 100 |
| G137D | | | | |
| Est23WT | 450 | 750 | — | — |
| Est23W251L | 980 | 550 | 1000 | 430 |
| E4 | 870 | 550 | ND | ND |

¹Not substantially different from values obtained using control cell extracts
²Not determined

Significantly, the 251 mutant with the highest deltamethrin activities was W251S, while W251L (highest for the other two pyrethroids), and W251G gave the lowest deltamethrin activities of the five 251 mutants. This suggests that accommodation of the α-cyano moiety of the leaving group may be the major impediment to efficient deltamethrin hydrolysis, sufficient to prevent any significant hydrolysis by wild type E3. Accommodation of substrate requires significantly different utilisation of space across the active site compared to other substrates, such that substitution of W251 in the acyl pocket with a smaller residue allows useful accommodation, particularly for αR isomers. Importantly, however, the details of the spatial requirements, and therefore the most efficacious mutants, differ from those for the other pyrethroids.

The activity of all enzymes with the 1S cis isomers of deltamethrin was dramatically less than with the corresponding isomer of NRDC157 lacking the α-cyano group. This dramatic influence of the α cyano group appears to be expressed with this 1S conformation at C1 of the cyclopropane group. With the exception of some of the least active mutants, activity against 1S cis isomers was again always greater with the αR than the αS conformation.

### Example 7

### General Discussion of Pyrethroid Experiments

Together, the permethrin and NRDC157 results for the 251 series mutants generate some quite strong and simple inferences about acyl binding constraints in E3/EST23. Overall, 251 replacements that should generate a more spacious acyl pocket do facilitate the accommodation/stabilisation of the bulky acyl groups of these substrates. These replacements are beneficial to the hydrolysis of all the isomers generated by the two stereocentres across the cyclopropane ring. While the trans isomers are strongly preferred by wild type enzyme, the mutants can also hydrolyse at least part of the cis isomer mix relatively well. However, within the cis isomers the improvements in the mutants is much more marked for the 1S cis isomers. The 1R cis isomers, which are the most problematic of all configurations for wild type enzyme, remain the most problematic for the mutants. Within the mutant series, the improved kinetics are not simply explained by the reduction in side chain size; the smallest substitution does not give the highest activities. Indeed the best kinetics are obtained with W251L, although Leu has the greatest side chain size of all the replacements tested, suggesting that its lipophilic nature plays a key role.

In contrast to the relatively simple and consistent patterns seen for permethrin and NRDC157, the deltamethrin results for the 251 series mutants are quite complex and difficult to interpret As might be expected from their enhanced kinetics for the other substrates, they do show overall better activities than wild type for the four cis deltamethrin isomers, albeit as with wild type they are much lower in absolute terms than for the other substrates. However, the preference for 1S over 1R isomers, which is so strong in respect of NRDC157, is weak at best in the deltamethrin data. On the other hand there is a clear trend across all the mutants for a preference for the αR over αS isomers. It is generally only of the order of 2:1, but notably it is opposite to the trend shown by wild type EST23. It is at first sight unexpected that these presumptive acyl binding pocket replacements should affect αR/αS stereopreferences because the latter apply to the α-cynano moiety in the (alcohol) leaving group of the substrate.

Overall the F309L data clearly show a major effect of this residue on the kinetics of pyrethroid hydrolysis. At one level there are parallels with the results for the W251 series mutants, both data sets showing enhanced kinetics consistent with expectations based on the provision of greater space in the acyl binding pocket. However, there are also important differences, with the W251 series disproportionately active for the cis vs trans isomers of permethrin and F309L disproportionately active with 1R vs 1S isomers of cis NRDC157. The replacements at the two sites also show strong interactions, consistent with them contributing to a shared structure and function in the acyl binding pocket. For example, both the disproportionate enhancement of the W251 mutants for cis permethrin and the disproportionate enhancement of F309L for 1R cis NRDC157 behave as dominant characters in the double mutant. The 251 and 309 mutants have quantitatively similar enhancing effects on activities and the same stereospecificities in respect of deltamethrin hydrolysis and the stereospecific differences seen with the smaller pyrethroids are not seen. However, we argue that the additional bulk of the αcyano moiety in its leaving group requires such a radical reallocation of space across the active site that the stereospecificities evident with the smaller pyrethroids are overridden.

### Example 8

### Fluorometric Determination of Lipase Activity

Assay for Lipase Activity

A fluorogenic assay was used to measure lipase activity of insect esterases or lipases, and mutants thereof. The fluorogenic substrate provides rapid reproducible methods for measuring enzymatic activity. Fatty acid esters (acylated) of 4-methylumbelliferone fluorophors are used as substrates for the identification of lipase activity. This assay uses the fluorophore 4-methylumbelliferyl palmitate (4-MU-palmitate) (structure provided below) and is a modification of the fluorometric esterase titration assay of Devonshire et al. (2002) and the method of Hamid et al. (1994) used for the rapid characterisation and identification of Mycobacteria.

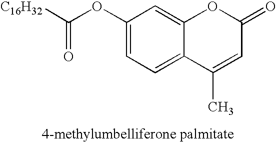

4-MU-palmitate is hydrolysed by a lipase to release the fluorescent 4-methylumbelliferone (4-MU), which can be measured by a fluorimeter.

A standard curve for 4-MU is prepared in each plate alongside the titrations. 25 μL 10⁻²M dMU stock (19.8 mg/10 ml in 100% ethanol) was diluted with 2.475 ml (3×825 μl) ethanol to give a 10⁻⁴M solution. This 10⁻⁴M solution was used to prepare a standard curve from 0 to 1.0 μM in 0.1M phosphate buffer pH 7.0 (plus 0.05% or 0.5% ultrapure Triton X-100 (TX100) if present in cell extracts). This was done by dispensing 25 μl, 20 μl, 15 μl, 10 μl, 5 μl, 0 μl (plus ethanol to 25 μl) into tubes and adding 2.475 ml phosphate buffer (or phosphate buffer containing TX100 if required), then adding 100 μl per well. This gives 0.2, 0.4, 0.6, 0.8 and 1.0 uM in 0.25% TX100.

The samples were read on a Fluorostar fluorometer (BMG LabTechnologies) alongside the following titration reactions using the basic settings: excitation—355 nm, emission—460 nm, gain—zero, 10 cycles of 180 secs with shaking before each cycle.

For the assay, 20 μl of 5×10⁻⁴ 4-MU-palmitate (in 100% acetone) was to the wells that require substrate (II & III as defined in Table 4 below) and air dried. For each enzyme to be assayed, 4 reactions were set up, first dispensing the buffer and then the cell sample. Cell extracts are 50 μl cell extract or cell supernatant and 50 μl phosphate buffer (0.1M) 0.05% TX-100. The final concentration of 4-MU-palmitate in the assay was 10⁻⁴M. Cell extracts should be added immediately before readings start.

TABLE 4

| | | | Contains |
|---|---|---|---|
| | Column of | | (P = 0.1 M phosphate |
| Identifier | plate | Reaction | buffer pH 7.0 +/− Triton) |
| I | 2 or 6 | Cell blank | 50 μl cell extract + 50 μl P_{nT} |
| II | 3 or 7 | Reaction | 50 μl cell extract + 50 μl P_{nT} |
| | | | (on dried 4-MU-palmitate) |
| III | 4 or 8 | 4-MU- | 50 μl P_{nT} + 50 μl P_{T} |
| | | palmitate | (on dried 4-MU-palmitate) |
| | | blank | |
| IV | 5 or 9 | Buffer | 50 μl P_{nT} + 50 μl P_{T} |
| | | blank | |

Corrected fluorescence (F_{corrected}) was calculated by the following equations.

For phosphate pH 7.0:
F_{corrected}=[(F_{II}−F_{I})/0.7]−F_{III}+2*F_{IV}]
For 0.05-0.5% TX100 in phosphate pH 7.0:
F_{corrected}=[(F_{II}−F_{I})/0.6]−F_{III}+2*F_{IV}]
where 0.6 and 0.7 are the quench correction factors for cell extracts at 10⁸ cells/ml, with and without TX100 respectively.
Results

The results of the lipase activity assay are provided in Table 5. Formal kinetic parameters from these data could not be calculated because of uncertainties around the solubility of the substrate. In general terms the data are most easily comparable to K_{cat} data. As such the values obtained show good lipase activity for the enzymes tested.

There is at least two orders of magnitude variation across the enzymes in 4UMP activity. However, there is no obvious correlation between 4UMP activity and naphthyl acetate, malathion or any pyrethroid hydrolytic activity across the various enzymes. Thus the data further demonstrate the versatility of the enzymes as a group in providing useful activities for a diverse range of substrates.

Two wild type enzymes, Myzus E4 and Drosophila alpha E2 give relatively high 4UMP activity, as do mutants of Lucilia E3 and Drosophila EST23. Thus the capability of hydrolysing 4UMP is distributed widely across the alpha carboxylesterase subclade.

There is at least one order of magnitude difference among the E3 mutants within each of the three active site subregions and in all three subregions there are mutants that are substantially better than wild type. As with the other substrates, mutations in all three subregions offer potential for improving lipase activity.

The W251L substitution clearly gives higher 4UMP activity in Myzus E4 and Drosophila EST23 but interestingly not in Lucilia E3. In the latter W251T is, however, clearly an improvement. F309L, also in the acyl pocket series, which was made because Leu is found at the equivalent position in some lipases, is also quite better than wild type.

F354L, in the anionic site, was also made because it is found in some lipases and it gives higher 4UMP activity as well. Comparative genomics would appear to be a promising approach to the design of enzymes with enhanced lipase activities. A few well chosen changes combined could make a very substantial change to the capabilities of esterases/lipases to hydrolyse hydrophobic (or conversely, hydrophilic) substrates.

TABLE 5
Esterase and lipase activities of natural and synthetic variants
of L. cuprina esterase E3, D. melanogaster EST23, Myzus persicae E4
and additional Drosophila carboxylesterases, as measured using
α-naphthyl acetate and 4-methyl umbelliferyl palmitate, respectively.

| | α-NA activity | | | 4-MU-palmitate activity |
|---|---|---|---|---|
| | Km | kcat | k_{cat}/Kₘ | moles 4MU produced per |
| Enzyme | (μM) | (sec⁻¹) | (M⁻¹sec⁻¹) | sec per mole of enzyme |
| E3WT | 71 | 248 | 3,500,000 | 0.0298 ± 0.0006¹ |

Oxyanion hole mutants

| | | | | |
|---|---|---|---|---|
| E3G137D | 27 | 24 | 890,000 | 0.1185 ± 0.0011 |
| E3G137R | 87 | 166 | 1,900,000 | 0.1452 ± 0.0221 |
| E3G137H | 114 | 55 | 480,000 | 0.0177 ± 0.0004 |
| E3G137E | 92 | 114 | 1,200,000 | 0.0199 ± 0.0045 |

Acyl binding pocket mutants

| | | | | |
|---|---|---|---|---|
| E3W251L | 188 | 145 | 770,000 | 0.0274 ± 0.0008 |
| E3W251S | 179 | 249 | 1,400,000 | 0.0482 ± 0.0070 |
| E3W251G | 80 | 294 | 3,700,000 | 0.0498 ± 0.0048 |
| E3W251T | 423 | 248 | 590,000 | 0.2481 ± 0.0254 |
| E3W251A | 251 | 503 | 2,000,000 | 0.0175 ± 0.0031 |
| E3F309L | 24 | 333 | 13,900,000 | 0.1210 ± 0.0032 |
| E3W251L/ | 153 | 112 | 730,000 | 0.0516 ± 0.0053 |
| F309L | | | | |
| E3W251L/ | 217 | 40 | 180,000 | 0.1421 ± 0.0122 |
| G137D | | | | |
| E3P250S/ | 47 | 57 | 1,200,000 | 0.0405 ± 0.0079 |
| W251L | | | | |

Anionic site mutants

| | | | | |
|---|---|---|---|---|
| E3Y148F | 27 | 129 | 4,800,000 | 0.0156 ± 0.0011 |
| E3Y148F/ | 34 | 23 | 680,000 | 0.0813 ± 0.0049 |
| G137D | | | | |
| E3E217M | 4 | 7 | 1,800,000 | 0.0864 ± 0.0053 |
| E3F354L | 36 | 20 | 570,000 | 0.1613 ± 0.0539 |
| E3F354W | 35 | 514 | 14,700,000 | 0.0459 ± 0.0027 |

EST23 enzymes

| | | | | |
|---|---|---|---|---|
| EST23WT | 82 | 276 | 3,400,000 | 0.0677 ± 0.0032 |
| EST23W251L | 24 | 26 | 1,100,000 | 0.4361 ± 0.0396 |
| EST23G137D | 111 | 34 | 310,000 | 0.1806 ± 0.0690 |

Myzus E4 enzymes

| | | | | |
|---|---|---|---|---|
| MpE4WT | 28 | 3 | 89,000 | 0.1051 ± 0.0033 |
| MpE4G113D | 59 | 3 | 51,000 | 0.0950 ± 0.0168 |
| MpE4W224L | 82 | 85 | 1,000,000 | 0.4890 ± 0.0071 |
| MpE4M190E | 56 | 3 | 54,000 | 0.0938² |
| MpE4G113D/ | 30 | 2 | 67,000 | 0.1410 ± 0.0652 |
| M190E | | | | |

Other Drosophila α-carboxylesterases

| | | | | |
|---|---|---|---|---|
| DmαE1 | 18 | 43 | 2,500,000 | 0.0522 ± 0.0127 |
| DmαE2 | 44 | 26 | 590,000 | 0.1993 ± 0.0583 |
| DmαE3 | 42 | 14 | 320,000 | 0.0200² |
| DmαE5 | 11 | 364 | 33,000,000 | 0.0026² |

¹Standard error of duplicate assays,
²Duplicates were not performed,
³Activity was detectable but too low to quantitate

### Example 9

### Bacterial Expression of Insect Esterases

Bacterial expression of E3 has proven to be successful in the GST fusion vector pGEX4T-1; the his-tag fusion vector pET146; and the vectors pTTQ18 and pKK223-3 that produce untagged protein. Successful expression has been observed in a wide range of E. coli strains including DH10B, TG1 and B121(DE3). These expression systems will be universally useful for all insect esterases or lipase, and mutants thereof, including mutants of E3 as they have proven successful for the wild-type E3 and 5 mutants.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Anderson, E. M., Larsson, K. M., & Kirk, O. (1998) Biocatalysis and Biotransformation 16, 181-204.
Berglund, P. (2001) Biomol Eng 18, 13-22.
Broomfield, C. A. (1999). Chemico-Biological Interactions 120: 251-256.
Campbell, P. M., Harcourt, R. L., Crone, E. J., Claudianos, C., Hammock, B. D., Russell, R. J. and Oakeshott, J. G. (2001) Insect Biochem Molec Biol 31:513-520.
Campbell, P. M., Newcomb, R. D., Russell, R. J. and Oakeshott, J. G. (1998a) Insect Biochem Molec Biol 28, 139-150.
Campbell, P. M., Yen, J. L., Masoumi, A., Russell, R. J., Batterham, P., McKenzie, J. A., and Oakeshott, J. G. (1998b) J. Econ. Entomol. 91:367-375.
Chaudhary, A. K., Lopez, J., Beckman, E. J., & Russell, A. J. (1997) Biotechnology Progress 13, 318-325.
Claudianos, C., Crone, E., Coppin, C., Russell, R. and Oakeshott, J. (2002) In: Marshall Clark, J. and Yamagushi, I., (Eds.) Agrochemical Resistance: Extent, Mechanism and Detection. (in press)
Claudianos, C., Russell, R. J. and Oakeshott, J. G. (1999) Insect Biochem Molec Biol 29, 675-86.
Cygler, M. and Schrag, J. D. (1997) Methods Enzymol 284, 3-27.
Derewenda, U., Brzozwski, A. M., Lawson, D. M., Derewenda, Z. S. (1992) Biochemistry 31:1532-1541.
Devonshire, A. L., Heidari, R., Bell, K. L., Campbell, P. M., Campbell, B. E., Odgers, W. A., Oakeshott, J. G. and Russell, R. J. Kinetic Efficiency of Mutant Carboxylesterases Implicated in Organophosphate Insecticide Resistance. (in preparation)
Devonshire, A. L. and Moores, G. D. (1982) Pestic. Biochem. Physiol. 18, 235-246.
Fournier, D., Bride, J.-M., Hoffmann, F. and Karch, F. (1992) J. Biol. Chem. 267, 14270-14274.
Gordon, R. K., Feaster, S. R., Russell, A. J., Lejeune, K. E., Maxwell, M. D., Lenz, D. E., Ross, M. and Doctor, B. P. (1999) Chem Biol Interact 14, 463-70.
Grochulski, P., Li, Y., Schrag, J. D., Bouthillier, F., Smith, P., Harrison, D., Rubin, B., Cygler, M. (1993). J Biol Chem 268, 12843-12847.
Hamid, M. E., Chun, J., Magee, J. G., Minnildn, D. E. and Goodfellow, M. (1994) Zentralbl. Bakteiol. 280, 476-487.
Harel, M., Kryger, G., Rosenberry, T. L., Mallender, W. D., Lewis, T., Fletcher, R. J., Guss, M., Silman, I. and Sussman, J. L. (2000) Protein Science 9, 1063-1072.
Hirohara, H. & Nishizawa, M. (1998) Biosci Biotechnol Biochem 62, 1-9.
Hirose, Y., Kariya, K., Nakanishi, Y., Kurono, Y., & Achiwa, K. (1995) Tetrahedron Letters 36, 1063-1066.
Jaeger, K. E. and Reetz, M. T. (1998) Trends in Biotechnology 16, 396-403.
Järv, J. (1984) Bioorganic Chemistry 12, 259-278.
Kazlauskas, R. & Bornscheuer, U. T. (1998), eds. Rehm, H. J. & Reed, G. (Wiley-VCH, Weinheim), pp. 37-191.
Koellner, G., Kryger, G., Millard, C. B., Silman, I., Sussman, J. L. and Steiner, T. (2000) The Journal of Molecular Biology 296, 713-735.
Lejuene, K. E., Wild, J. R. and Russell, A. J. (1998) Nature 395, 27-8.
Liebeton, K., Zonta, A., Schimossek, K., Nardini, M., Lang, D., Dijkstra, B. W., Reetz, M. T., & Jaeger, K. E. (2000) Chemistry & Biology 7, 709-718.
Liese, A. & Filho, M. V. (1999) Current Opinion in Biotechnology 10, 595-603.
Martinez, C., Nicolas, A., van Tilbeurgh, H., Egloff, M. P., Cudrey, C., Verger, R. and Cambillau, C. (1994) Biochemistry 33, 83-9.
Myers, M. A., Healy, M. J. and Oakeshott, J. G. (1993) Biochem Genet 31, 259-78.
Nair, H. K., Seravalli, J., Arbuckle, T. and Quinn, D. M. (1994) Biochemistry 33, 8566-76.
Needleman, S. B. and Wunsch, C. D. (1970) J Mol Biol 48, 443-53.
Newcomb, R. D., Campbell, P. M., Ollis, D. L., Cheah, E., Russell, R. J., & Oakeshott, J. G. (1997) Proc Natl Acad Sci USA 94, 7464-8.
Oakeshott, J. G., Claudianos, C., Russell, R. J., & Robin, G. C. (1999) BioEssays 21, 1031-42.
Oakeshott, J. G., van Papenrecht, E. A., Boyce, T. M., Healy, M. J. and Russell, R. J. (1993) Genetica 90, 239-268.
Ordentlich, A., Barak, D., Kronman, C., Ariel, N., Segal, Y., Velan, B, and Shaferman, A. (1998) The Journal of Biological Chemistry 273, 19509-19517.
Ordentlich, A., Barak, D., Kronman, C., Ariel, N., Segal, Y., Velan, B. and Shaferman, A. (1995) The Journal of Biological Chemistry 270, 2082-2091.
Ordentlich, A., Barak, D., Kronman, C., Ariel, N., Segal, Y., Velan, B. and Shaferman, A. (1996) The Journal of Biological Chemistry 271, 11953-11962.
Ordentlich, A., Barak, D., Kronman, C., Flashner, Y., Leitner, M., Segal, Y., Ariel, N., Cohen, S., Velan, B. and Shafferman, A. (1993) The Journal of Biological Chemistry 268, 17083-17095.
Pandey, A., Benjamin, S., Soccol, C. R., Nigam, P., Krieger, N., & Soccol, V. T. (1999) Biotechnology and Applied Biochemistry 29, 119-131.
Patten, P. A., Howard, R. J. and Stemmer, W. P. (1997) Curr Opin Biotechnol 8, 724-33.
Petrikovics, I., Cheng, T. C., Papahadjopoulos, D., Hong, K., Yin, R., DeFrank, J. J., Jaing, J., Zong, Z. H., McGuinn, W. D., Sylvester, D., Pei, L., Madec, J., Tamulinas, C., Jaszberenyi, J. C., Barcza, T. and Way, J. L. (2000a) Toxicol Sci 57, 16-21.
Petrikovics, I., McGuinn, W. D., Sylvester, D., Yuzapavik, P., Jaing, J., Way, J. L., Papahadjopoulos, D., Hong, K., Yin, R., Cheng, T. C., and DeFrank, J. J. (2000b) Drug Delivery 7: 83-89.
Phythian, S. J. (1998) 4 Esterases. Kelly D. R. Biotransformations I. 194-241.
Qian, N. and Kovach, I. M. (1993) FEBS Lett 336, 263-6.
Robin, C., R. J. Russell, K. M. Medveczky, and J. G. Oakeshott. (1996) J Mol Evol 43: 241-52.
Rubio, E., Fernandez-Mayorales, A. and Klibanov., A. M. (1991) J Am Chem Soc 113, 695-696.
Satoh, T. and Hosokawa, M. (1998) Annu. Rev. Pharmacol. Toxicol. 38, 257-288.
Scheib, H., Pleiss, J., Stadler, P., Kovac, A., Potthoff, A. P., Haalck, L., Spener, F., Paltauf, F., & Schmid, R. D. (1998) Protein Engineering 11, 675-682.
Shafferman, A., Velan, B., Orentlich, A., Kronman, C., Grosfeld, H., Leitner, M., Flachner, Y., Cohen, S., Barak, D. and Ariel, N. (1992) EMBO J. 11, 3561-3568.
Stryer, L. (1981) Biochemistry. p 115, W.H. Freeman, San Francisco.
Sussman, J. S., Harel, M., Frolov, F., Oefner, C., Goldman, A., Toker, L. and Silman, I. (1991) Science 253, 872-879.
Svendsen, A. (2000) Biochim Biophys Acta 1543, 223-238.
Thomas, B. A., Church, W. B., Lane, T. R. and Hammock, B. D. (1999) Proteins 34,184-96.
Villatte, F., Ziliani, P., Marcel, V., Menozzi, P. and Fournier, D. (2000) Pesticide Biochemistry and Physiology 95-102.
Villeneuve, P., Muderhwa, J. M., Graille, J., & Haas, M. J. (2000) Journal of Molecular Catalysis B.: Enzymatic 9, 113-148.
Walsh, S. B., Dolden, T. A., Moores, G. D., Kristensen, M., Lewis, T., Devonshire, A. L. and Williamson, M. S. (2001) Biochem J 359, 175-81.
Yao, H., Chunling, Q., Williamson, M. S. and Devonshire, A. L. (1997) Clin. J. Biotechnol. 13: 177-183.
Zhu, Y.-C., Dowdy, A. K. and Baker, J. E. (1999) Insect Biochem Molec. Biol. 29: 417-425.

1. An enzyme-based biocatalysis process, wherein the enzyme is an insect esterase or lipase, or mutant thereof.

2. A process according to claim 1, wherein the esterase or lipase-based biocatalysis comprises or includes to the scheme: 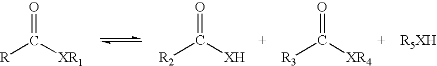
wherein
R, R₂ and R₃ are the same moiety Z, or
R is a mixture of stereoisomers of the moiety Z, R₂ is an stereoisomer of the moiety Z and R₃ is a mixture of stereoisomers enriched in another stereoisomer of moiety Z;
R₁, R₄ and R₅ are the same moiety Y, or
R₁ is a mixture of stereoisomers of the moiety Y, R₅ is one stereoisomer of the moiety and R₄ is a mixture of enantiomers enriched in another stereoisomer of moiety Y;
moieties Z and Y may be individually selected from a substituted or unsubstituted hydrocarbon moiety optionally interrupted by one of more heteroatoms; and
X is a nucleophilic group.

3. A process according to claim 2, wherein the stereoisomers are enantiomers or positional stereoisomers.

4. A process according to claim 2 when carried out under conditions in which the forward reaction predominates.

5. A process according to claim 1 when used for chemo-, regio- or stereo-selective hydrolysis of at least one acid ester.

6. A process according to claim 5, wherein the ester is an insecticide containing an ester group.

7. A process according to claim 6, wherein the ester is a pyrethroid.

8. A process according to claim 7, wherein the pyrethroid is selected from the group consisting of: permethrin, cyloprothrin, fenvalerate, esfenvalerate, flucythrinate, fluvalinate, fenpropathrin, d-fenothrin, cyfenothrin, allethrin, cypermethrin, deltamethrin, tralomethrin, tetramethrin, resmethrin and cyfluthrin.

9. A process according to claim 5, wherein the ester is a fatty acid ester.

10. A process according to claim 5, when used for resolution of a stereoisomer from a mixture of stereoisomers of a carboxylic acid ester.

11. A process according to claim 1 for optical resolution of a mixture of a (R)-ester compound and a (S)-ester compound comprising the steps of:
(a) contacting an insect esterase or lipase, or mutant thereof, with the mixture to obtain an optically acid compound or an optically active alcohol compound by stereoselectively hydrolyzing one of the (R)-ester compound and the (S)-ester compound; and
(b) recovering an optically active compound selected from the group consisting of the optically active acid compound, the optically active alcohol compound and the optically active ester that is not hydrolysed.

12. A process according to claim 1, when used for producing an optically active acid.

13. A process according to claim 12, wherein the optically active acid is a pyrethroid acid.

14. A process according to claim 13, wherein the pyrethroid acid is selected from the group consisting of: permethrin, cyloprothrin, fenvalerate, esfenvalerate, flucythrinate, fluvalinate, fenpropathrin, d-fenothrin, cyfenothrin, allethrin, cypermethrin, deltamethrin, tralomethrin, tetramethrin, resmethrin and cyfluthrin.

15. A process according to claim 1, wherein the optically active acid is a cyclopropane carboxylic acid.

16. A process according to claim 1 when used for the production of an optically active alcohol.

17. A process according to claim 16, wherein the optically active alcohol is a pyrethroid alcohol.

18. A process according to claim 17, wherein the pyrethroid alcohol is the alcohol of a pyrethroid selected from the group consisting of: permethrin, cyloprothrin, fenvalerate, esfenvalerate, flucythrinate, fluvalinate, fenpropathrin, d-fenothrin, cyfenothrin, allethrin, cypermethrin, deltamethrin, tralomethrin, tetramethrin, resmethrin and cyfluthrin.

19. A process according to claim 1 which is a transesterification or an interesterification reaction.

20. A process according to claim 1 when used for the modification of vegetable oils or fats suitable for use in emulsions and other fat-based food products.

21. A process according to claim 20, wherein the food product is selected from the group consisting of: margarine, artificial creams and ice creams.

22. A process according to claim 1 when used for the production of a polymer.

23. A process according to claim 22, wherein the polymer is a polyester.

24. A process according to claim 23, wherein the polyester is produced by successive esterification and transesterification of di functional esters and alcohols, self-condensation of di functional monomers, and ring opening polymerisation of lactones

25. A process according to claim 1 carried out under conditions in which the back reaction predominates.

26. A process according to claim 25 when used for the acylation of a substrate.

27. A process according to claim 1, wherein the insect esterase or lipase is an α-carboxylesterase.

28. A process according to claim 27, wherein the mutant insect esterase or lipase is an α-carboxylesterase, and has a mutation(s) in an oxyanion hole, acyl binding pocket or anionic site regions of an active site of the esterase or lipase, or any combination thereof.

29. A process according to claim 28, wherein the mutant insect esterase or lipase is selected from the group consisting of: E3G137R, E3G137H, E3W251L, E3W251S, E3W251G, E3W251T, E3W251A, E3W251L/F309L, E3W251L/G137D, E3W251L/P250S, E3F309L, E3Y148F, E3E217M, E3F354W, E3F354L, and EST23W251L.

30. A process according to claim 27, wherein the α-carboxylesterase, or mutant thereof, comprises a sequence selected from the group consisting of:
i) a sequence as shown in SEQ ID NO:1,
ii) a sequence as shown in SEQ ID NO:2,
iii) a sequence as shown in SEQ ID NO:3,
iii) a sequence which is at least 40% identical to any one of i) to iii) which is capable of hydrolysing a hydrophobic ester.

31. A process according to claim 30, wherein the sequence is at least 80% identical to i) or ii).

32. A process according to claim 30, wherein the sequence is at least 90% identical to i) or ii).

33. A process according to claim 1, wherein the insect esterase or lipase, or mutant thereof, is expressed from a recombinant host cell.

34. A process according to claim 33, wherein the host cell is a bacterial cell.

35. A process according to claim 33, wherein the host cell is a fungal cell.

36. A method for generating and selecting an enzyme that hydrolyses a hydrophobic ester, the method comprising
(i) introducing one or more mutations into an insect esterase or lipase, or an insect esterase or lipase that has already been mutated, and
(ii) determining the ability of the mutant insect esterase or lipase to hydrolyse the hydrophobic ester.

37. The method of claim 36, wherein the hydrophobic ester is a fatty acid ester.

38. The method of claim 36, wherein the one or more mutations enhances hydrolytic activity and/or alters the stereospecificity of the esterase or lipase.

39. The method according to claim 36, wherein the insect esterase or lipase is an α-carboxylesterase.

40. The method of claim 39, wherein the α-carboxylesterase has a sequence selected from the group consisting of:
i) a sequence as shown in SEQ ID NO:1,
ii) a sequence as shown in SEQ ID NO:2,
iii) a sequence as shown in SEQ ID NO:3, and
iv) a sequence which is at least 40% identical to any one of i) to iii).

41. The method of claim 40, wherein the sequence is at least 80% identical to any one of i) to iii).

42. The method of claim 40, wherein the sequence is at least 90% identical to any one of i) to iii).

43. The method of claim 36, wherein the one or more mutations are within a region of the esterase or lipase selected from the group consisting of: oxyanion hole, acyl binding pocket and anionic site.

44. The method of claim 36, wherein the mutation is a point mutation.

45. The method of claim 44, wherein the insect esterase or lipase that has already been mutated is selected from the group consisting of: E3G137R, E3G137H, E3W251L, E3W251S, E3W251G, E3W251T, E3W251A, E3W251L/F309L, E3W251L/G137D, E3W251L/P250S, E3F309L, E3Y148F, E3E217M, E3F354W, E3F354L, and EST23W251L.

46. A method for generating and selecting an insect α-carboxylesterase that hydrolyses an ester, the method comprising
(i) introducing one or more mutations into an insect α-carboxylesterase, or an insect α-carboxylesterase that has already been mutated, and
(ii) determining the ability of the mutant insect α-carboxylesterase to hydrolyse the ester.

47. An enzyme obtained by a method according to claim 36.


SEQUENCE LISTING (4 sequences)

SEQ ID NO: 1 - protein, 570 aa, Lucilia cuprina
MNFNVSLMEKLKWKIKCIENKFLNYRLTTNETVVAETEYGKVKGVKRLTVYDDSYYSFEGIPYAQPPVGELRFKAPQRPTPWDGVRDCCNHKDKSVQVDFITGKVCGSEDCLYLSVYTNNLNPETKRPVLVYIHGGGFIIGENHRDMYGPDYFIKKDVVLINIQYRLGALGFLSLNSEDLNVPGNAGLKDQVMALRWIKNNCANFGGNPDNITVFGESAGAASTHYMMLTEQTRGLFHRGILMSGNAICPWANTQCQHRAFTLAKLAGYKGEDNDKDVLEFLMKAKPQDLIKLEEKVLTLEERTNKVMFPFGPTVEPYQTADCVLPKHPREMVKTAWGNSIPTMMGNTSYEGLFFTSILKQMPMLVKELETCVNFVPSELADAERTAPETLEMGAKIKKAHVTGETPTADNFMDLCSHIYFWFPMHRLLQLRFNHTSGTPVYLYRFDFDSEDLINPYRIMRSGRGVKGVSHADELTYFFWNQLAKRMPKESREYKTIERMTGIWIQFATTGNPYSNEIEGMENVSWDPIKKSDEVYKCLNISDELKMIDVPEMDKIKQWESMFEKHRDLF

SEQ ID NO: 2 - protein, 572 aa, Drosophila melanogaster
MNKNLGFVERLRGRLKTIEHKVQQYRQSTNETVVADTEYGQVRGIKRLSLYDVPYFSFEGIPYAQPPVGELRFKAPQRPIPWEGVRDCSQPKDKAVQVQFVFDKVEGSEDCLYLNVYTNNVKPDKARPVMVWIHGGGFIIGEANREWYGPDYFMKEDVVLVTIQYRLGALGFMSLKSPELNVPGNAGLKDQVLALKWIKNNCASFGGDPNCITVFGESAGGASTHYMMLTDQTQGLFHRGILQSGSAICPWAYNGDITHNPYRIAKLVGYKGEDNDKDVLEFLQNVKAKDLIRVEENVLTLEERMNKIMFRFGPSLEPFSTPECVISKPPKEMMKTAWSNSIPMFIGNTSYEGLLWVPEVKLMPQVLQQLDAGTPFIPKELLATEPSKEKLDSWSAQIRDVHRTGSESTPDNYMDLCSIYYFVFPALRVVHSRHAYAAGAPVYFYRYDFDSEELIFPYRIMRMGRGVKGVSHADDLSYQFSSLLARRLPKESREYRNIERTVGIWTQFAATGNPYSEKINGMDTLTIDPVRKSDEVIKCLNISDDLKFIDLPEWPKLKVWESLYDDNKDLLF

SEQ ID NO: 3 - protein, 552 aa, Myzus persicae
MKNTCGILLNLFLFIGCFLTCSASNTPKVQVHSGEIAGGFEYTYNGRKIYSFLGIPYASPPVQNNRFKEPQPVQPWLGVWNATVPGSACLGIEFGSGSKIIGQEDCLFLNVYTPKLPQENSAGDLMNVIVHIHGGGYYFGEGILYGPHYLLDNNDFVYVSINYRLGVLGFASTGDGVLTGNNGLKDQVAALKWIQQNIVAFGGDPNSVTITGMSAGASSVHNHLISPMSKGLFNRAIIQSGSAFCHWSTAENVAQKTKYIANLMGCPTNNSVEIVECLRSRPAKAIAKSYLNFMPWRNFPFTPFGPTVEVAGYEKFLPDIPEKLVPHDIPVLISIAQDEGLIFSTFLGLENGFNELNNNWNEHLPHILDYNYTISNENLRFKTAQDIKEFYFGDKPISKETKSNLSKMISDRSFGYGTSKAAQHIAAKNTAPVYFYEFGYSGNYSYVAFFDPKSYSRGSSPTHGDETSYVLKMDGFYVYDNEEDRKMIKTMVNIWATFIKSGVPDTENSEIWLPVSKNLADPFRFTKITQQQTFEAREQSTTGIMNFGVAYH

SEQ ID NO: 4 - protein, 576 aa, Torpedo californica
ADDDSELLVNTKSGKVMRTRIPVLSSHISAFLGIPFAEPPVGNMRFRRPEPKKPWSGVWNASTYPNNCQQYVDEQFPGFPGSEMWNPNREMSEDCLYLNIWVPSPRPKSATVMLWIYGGGFYSGSSTLDVYNGKYLAYTEEVVLVSLSYRVGAFGFLALHGSQEAPGNMGLLDQRMALQWVHDNIQFFGGDPKTVTLFGESAGRASVGMHILSPGSRDLFRRAILQSGSPNCPWASVSVAEGRRRAVELRRNLNCNLNSDEDLIQCLREKKPQELIDVEWNVLPFDSIFRFSFVPVIDGEFFPTSLESMLNAGNFKKTQILLGVNKDEGSFFLLYGAPGFSKDSESKISREDFMSGVKLSVPHANDLGLDAVTLQYTDWMDDNNGIKNRDGLDDIVGNHNVICPLMHFVNKYTKFGNGTYLYFFNHRASNLVWPEWMGVIHGYEIEFVFGLPLVKELNYTAEEEALSRRIMHYWATFAKTGNPNEPHSQESKWPLFTTKEQKFIDLNTEPIKVHQRLRVQMCVFWNQFLPKLLNATETIDEAERQWKTEFHRWSSYMMHWKNQFDQYSRHENCAEL